(19) United States
(12) Patent Application Publication
(10) Pub. No.: US 2018/0100134 A1
(43) Pub. Date: Apr. 12, 2018

(54) CANCER CELL ENRICHMENT SYSTEM

(71) Applicant: XCELL BIOSCIENCES, INC., San Francisco, CA (US)

(72) Inventors: James LIM, Oakland, CA (US); Luke CASSEREAU, Emeryville, CA (US)

(21) Appl. No.: 15/566,337

(22) PCT Filed: Apr. 15, 2016
(86) PCT No.: PCT/US16/27881

§ 371 Date: Oct. 13, 2017

Related U.S. Application Data
(60) Provisional application No. 62/149,268, filed on Apr. 17, 2015.

(51) Int. Cl. C12M 1/00; C12N 5/09; C12M 1/34; C12M 1/36

(52) U.S. Cl. CPC C12M 41/14; C12N 5/0693; C12M 41/48; C12M 41/40; C12M 41/34

(57) ABSTRACT

The present invention describes a method for the propagation of a subpopulation of cells. The subpopulation of cells can be grown in specific culture conditions to promote growth and enrichment of the subpopulation of cells. The method can be used to determine biomarkers, and guide treatment of cancer patients.

### CROSS REFERENCE

This Application claims the benefit of U.S. Provisional Application No. 62/149,268, filed Apr. 17, 2015, which is incorporated herein by reference in its entirety.

### BACKGROUND

Cell enrichment systems can be used to enrich and isolate different populations of cells. The cells can include, for example, cancer cells, circulating tumor cells, stem cells, or immune cells. Isolation and characterization of different cell types that are induced through a cell enrichment system can be used to understand tumor etiology, the biology of metastasis, stem cell differentiation, immune cell proliferation, and to provide a biomarker for tumor progression.

### INCORPORATION BY REFERENCE

Each patent, publication, and non-patent literature cited in the application is hereby incorporated by reference in its entirety as if each was incorporated by reference individually.

### SUMMARY OF THE INVENTION

In some embodiments, the invention provides a cell culture incubator, wherein the cell culture incubator comprises: a) an enclosed environmental chamber; and b) a control unit, wherein the control unit is operably linked to the enclosed environmental chamber, wherein the control unit comprises a computer program product comprising a computer-readable medium having computer-executable code encoded therein, the computer-executable code adapted to encode: (i) an oxygen level module, wherein the oxygen level module is encoded to regulate an oxygen level of the enclosed environmental chamber, wherein the oxygen level module is encoded to control the removal of oxygen in the enclosed environmental chamber to generate a hypoxic oxygen level within the enclosed environmental chamber; (ii) a pressure module, wherein the pressure module is encoded to regulate the pressure of the enclosed environmental chamber, wherein the pressure module controls the addition of gas to generate a positive pressure condition in the enclosed environmental chamber; (iii) a temperature module, wherein the temperature module is encoded to regulate the temperature of the enclosed environmental chamber; and (iv) a humidity module, wherein the humidity module is encoded to regulate the humidity of the enclosed environmental chamber, wherein each of the oxygen level, pressure, temperature, and humidity mimics an in vivo microenvironment for a cell, wherein the cell culture incubator reaches each of an instructed oxygen level, pressure, temperature, and humidity within about 20 minutes of receiving an input of each of the instructed oxygen level, pressure, temperature, and humidity.

### BRIEF DESCRIPTION OF THE FIGURES

FIG. 1 is an immunofluorescence image of a representative CTC cluster.

FIG. 2 is an illustrative workflow for enrichment of a target subpopulation of cells.

FIG. 3 depicts enrichment and propagation of a target subpopulation of cells using a method of the invention.

FIG. 4 is an illustrative computer system to be used with a method of the invention.

FIG. 5 depicts results of biomarker assessment in CTCs from prostate cancer cells.

FIG. 6 shows results of biomarker determination for prostate cancer cells.

FIG. 7 displays results of mRNA sequencing analysis for the nerve growth factor signaling pathway.

FIG. 8 displays results of mRNA sequencing analysis for the Aurora A signaling pathway.

FIG. 9 displays results of mRNA sequencing analysis for the Kit receptor signaling pathway.

FIG. 10 is an immunofluorescence image for EPCAM expression in a PDAC CTC.

FIG. 11 depicts results of NANOG signaling pathway expression in PDAC versus mCRPC CTCs.

FIG. 12 depicts results of Wnt signaling pathway expression in PDAC versus mCRPC CTCs.

FIG. 13 displays results of SNP and INDEL analysis for CTCs.

FIG. 14 displays results of SNP and INDEL analysis for CTCs.

FIG. 15 depicts an illustrative user interface for a system of the invention.

FIG. 16 depicts an illustrative user interface for a system of the invention.

FIG. 17 depicts an illustrative user interface for a system of the invention.

FIG. 18 depicts an illustrative user interface for a system of the invention.

FIG. 19 displays an illustrative cell culture incubator of the invention.

FIG. 20 displays a door configuration of a cell culture incubator of the invention.

FIG. 21 displays a door configuration of a cell culture incubator of the invention.

FIG. 22 depicts a door heater of a cell culture incubator of the invention.

FIG. 23 depicts increased cellular transfection efficiency using green fluorescent protein.

### DETAILED DESCRIPTION

### Method of the Invention.

A method of the present invention can be used to isolate CTCs, and other target cell subpopulations, from a biological sample. A method of the invention can be used, for example, to isolate cell populations, selectively separate cell populations, maintain cells in a differentiated or an undifferentiated state, forcibly differentiate cells, enrich cell populations, expand cell populations (through proliferation or selective enrichment), modulate functions of cell populations, modulate morphology of cell populations, modulate epigenetic characteristics, and modulate gene and protein expression profiles. A method of the invention can be used in, for example, primary cells, cell lines, or microbial communities.

Target cell subpopulations can include, for example, CTCs, cancer stem cells (CSCs), hematopoietic stem cells (HSCs), endothelial progenitor cells (EPCs), pre-cancerous cells, stem cells, fetal stem cells, undifferentiated stem cells, fetal cells, bone marrow cells, progenitor cells, foam cells, mesenchymal cells, epithelial cells, epithelial progenitor cells, endothelial cells, endometrial cells, trophoblasts, cancer cells, red blood cells, white blood cells, immune system cells, connective tissue cells, hepatocytes, neurons, induced pluripotent stem (IPS) cells, or any combination thereof.

A method of the invention can be used, for example, to maintain neuronal cells in culture, to maintain hepatocytes in culture, and toxicity screening. The invention can be used to differentiate IPS cells and stems cell into, for example, cells of the mesoderm, ectoderm, and endoderm. A method of the invention can be used to differentiate cells into neurons, cardiomyocytes, hepatocytes, hematopoietic stem cells, osteoblasts, osteoclasts, epithelial cells, endothelial cells, astrocytes, adipocytes, immune cells, mast cells, erythrocytes, oocytes, or spermatocytes.

CTCs can be composed of heterogeneous clusters of cancer and immune cells in vivo and can display differential expression of immunomodulatory and stem cell signaling pathway in vitro.

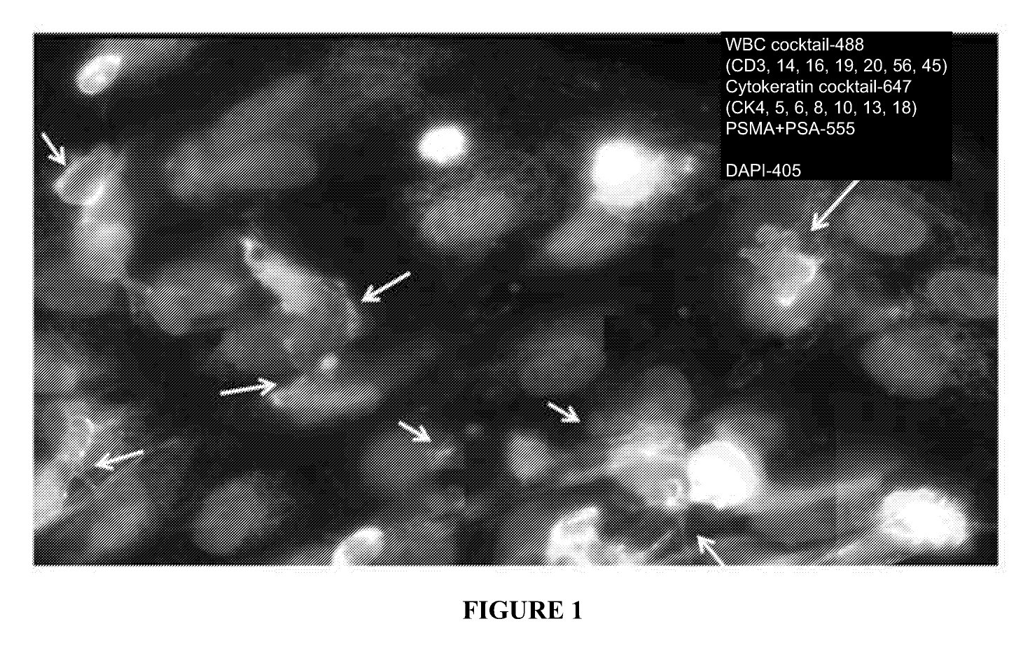

FIG. 1 displays immunofluorescence (IF) for a circulating tumor cell cluster from a subject with castration-resistant prostate cancer (CRPC). The IF image demonstrates that CTCs can contain both cancer and immune cells. The DAPI staining (indicated by oval-shaped cell staining) indicates the nuclei of the cells. A white blood cell (WBC) antibody cocktail can be used to detect, for example, CD3, CD14, CD16, CD19, CD20, CD45, and CD56, in the CTC cluster (white arrows in FIG. 1). A cytokeratin antibody cocktail can be used to detect, for example, CK4, CK5, CK6, CK8, CK10, CK13, and CK18 in the CTC cluster (fibrillar staining seen in and around oval-shaped nuclei in FIG. 1). The bright white staining, and the fibrillar staining around the oval-shaped nuclei, indicates prostate-specific membrane antigen (PSMA) and prostate-specific antigen (PSA) proteins. The numbers in the legend indicate the wavelength (nm) used for excitation of the stains.

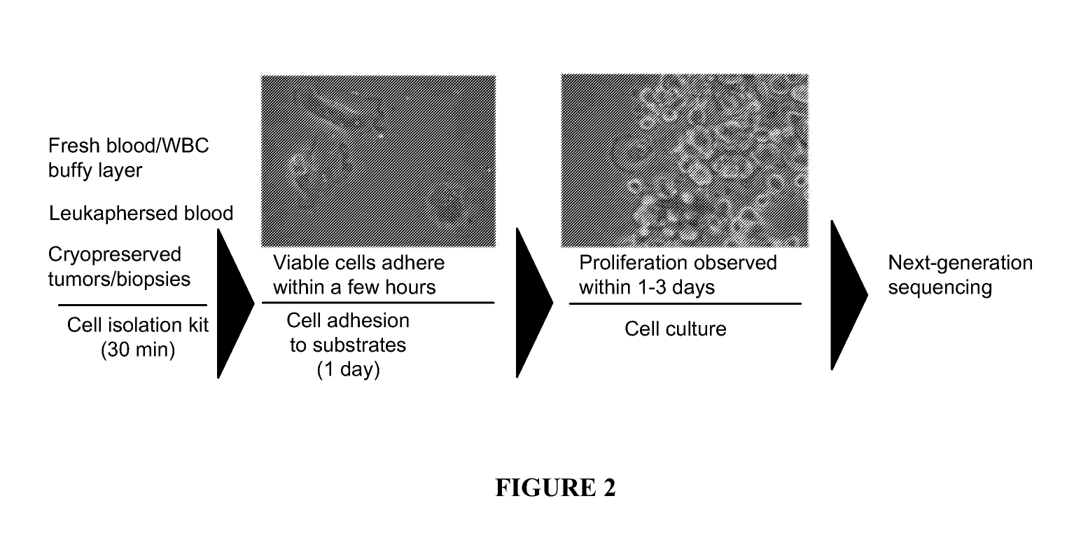

FIG. 2 depicts an illustrative workflow that can be used to obtain an enriched population of cells from a heterogeneous cell population. The sources of cells used in a method of the invention can include, for example, fresh blood, white blood cells (WBC), the buffy layer of centrifuged blood, cryopreserved tumors and biopsies, samples for leukapheresis, fine needle aspirates, fresh biopsies, urine, or fecal matter. After obtaining a sample from a subject, the sample can be prepared for use in a cell isolation kit to separate, for example, a blood sample into plasma, white blood cells and platelets, and red blood cells. CTCs can be found in the white blood cell and platelet fraction of centrifuged blood. The cell isolation step can be about 30 minutes long. After the heterogeneous cell population has been isolated, the cells can be applied to the enrichment medium for propagation and enrichment of viable CTC colonies. The adhesion of the cells to the substrate can take from a few hours to about one day. Proliferation during culture of the cells can be observed within about 1 to 3 days after adherence, after which next-generation sequencing (NGS) can be performed on the cell colonies for the markers of interest. Next-generation sequencing methods can include, for example, whole genome sequencing, whole genome resequencing, whole exome sequencing, whole transcriptome mRNA sequencing, ChIP-sequencing, and bioinformatics.

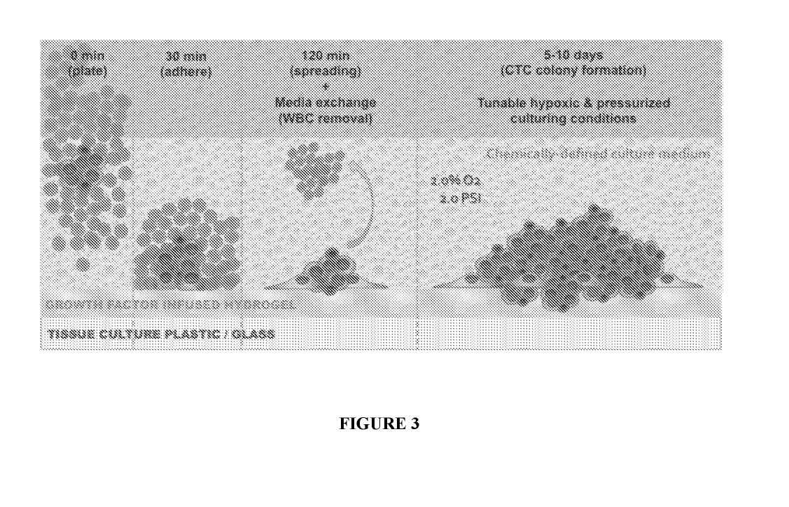

FIG. 3 depicts adherence and propagation of a collected heterogeneous cell population from a subject. First, the cells can be applied to a plate, which can be coated with a substance, such as a growth factor-infused hydrogel. After about 30 minutes, the cells are adhered to the plate. Over the next two hours, the cells spread across the plate, and the growth medium is exchanged allowing for removal of any remaining white bloods cells (WBCs). The media is replaced with a chemically-defined culture medium to promote the growth of CTC colonies. The cells can be grown in an environment that can be adjusted to mimic, for example, the tumor microenvironment, via changes in oxygen or pressure levels to obtain viable CTCs. In some embodiments, the cells are grown in hypoxic conditions.

The present invention can use a substrate to capture target cell subpopulations from a sample. The heterogeneous cell population can be applied to, for example, a culture dish coated with a substrate that can promote growth and enrichment of the target cell population. The target cell subpopulation can adhere to the substrate with higher affinity than other cells, for example, white blood cells. Cells that do not adhere to the substrate can be washed away with media or maintained in culture. Once adhered, the cells can spread and begin dividing on the substrate.

The substrate can comprise, for example, 1, 2, 3, 4, or 5 layers. The distance between two substrates layers may range from about 0.001 to about 20 mm, about 1 to about 10 mm, or about 1 to about 5 mm and each layer can be about 0.001, about 1, about 2, about 3, about 4, about 5, about 6, about 7, about 8, about 9, about 10, about 12, about 15, about 17, or about 20 mm.

The cells can be plated on a material made of, for example, plastic, glass, gelatin, polyacrylamide, or any combination thereof. The dishes used to the plate the cells can be, for example, microscope slides, culture plates, culture dishes, Petri dishes, microscope coverslips, an enclosed environmental chamber, a sealed culture dish, or multi-well culture dishes.

The binding surface layer of the substrate can be the portion of the substrate that is in contact with the captured cells. In some instances, the binding surface layer is the only layer, adjacent to the base layer, or separated from the base layer by one or more middle layers.

The binding surface layer of the substrate can comprise, for example, cell monolayers, cell lysates, biological materials associated with the extracellular matrix (ECM), gelatin, or any combination thereof.

Biological materials associated with the ECM can include, for example, collagen type I, collagen type IV, laminin, fibronectin, elastin, reticulin, vimentin, hygroscopic molecules, glycosaminoglycanse, proteoglycans, roteoglycans, glycocalyx, bovine serum albumin, human serum albumin, Poly-L-lysine, Poly-D-lysine, or Poly-L-ornithine. The gelatin can be from an animal source, for example, the gelatin can porcine or bovine.

The monolayer of cells used in the substrate can be, for example, mammalian cells, endothelial cells, vascular cells, venous cells, capillary cells, human umbilical vein endothelial cells (HUVEC), human lung microvascular endothelial cells (HLMVEC), human keratinocytes, human mesenchymal stem cells, human bone marrow stromal cells, and human astroglial cells. The cell lines can be obtained from a primary source or from an immortalized cell line. The monolayer of cells can be irradiated by ultraviolet light or X-ray sources to cause senescence of cells. The monolayer can also contain a mixture of one or more different cell types. The different cell types may be co-cultured together. One non-limiting example of co-culture is a combination of primary human endothelial cells co-cultured with transgenic mouse embryonic fibroblasts mixed to form a monolayer.

The binding surface layer of the substrate can contain, for example, a mixture of intracellular components. One method that can be used to obtain a mixture of intracellular components is lysis of the cells and collection of the cytosolic and cytoskeletal components. The lysed cells may be primary or immortalized. The lysed cells can be from either mono- or co-cultures.

The binding surface layer of the substrate can contain biological materials associated with the extracellular matrix (ECM) or binding moieties such as hyaluronic acid hydrogels. For example, gelatin can be mixed directly with cells, binding moieties, biological materials associated with the ECM, or any combination thereof, to make a binding surface layer for the substrate. For example, the binding surface layer can comprise a gelatin mixed with a collagen.

The substrate can have one or more middle layers. The middle layer of the substrate can be one or more monolayers of cells. The cells of the monolayer can be of varying origin. For example, the middle layer of the substrate can be made by growing a confluent monolayer of mouse embryonic fibroblasts on the base layer and then growing another layer of cells, for example, the binding surface layer, on top of the confluent mouse embryonic fibroblasts.

A feeder layer can be used in the substrate for growth and enrichment of the target cell subpopulation. A feeder layer can sit adjacent to a base layer and can be separated from the binding surface layer of the substrate. The feeder layer can be a monolayer of feeder cells. The cells of the monolayer can be of varying origin. For example, the feeder layer can be made by growing a monolayer of human endothelial cells or mouse embryonic fibroblasts on a base layer.

Conjugation of layers of the substrate can be done by allowing cells to grow in a monolayer on top of the base layer or middle layer. Conjugation of layers can also be done by pre-treating the surface with a surface of either net positive, net negative, or net neutral charge. The conjugation procedure can be aided by chemical moieties, linkers, protein fragments, nucleotide fragments, or any combination thereof.

The configuration and composition of the substrate can be tailored for enrichment of a particular target cell subpopulation. The composition of the substrate can vary based on, for example, patient type, cancer type, stage of cancer, patient medical history, and genomic and proteomic analysis of the patient tumor.

The enrichment media used for growing the cells can be supplemented or made with culture media that has been collected from cell cultures, blood plasma, or any combination thereof. The enrichment media can be, for example, Plating Culture Medium, Type R Long Term Growth Medium, Type DF Long Term Growth Medium, Type D Long Term Growth Medium, and MEF—Enrichment Medium, or any combination thereof. The enrichment medium can contain, for example, a primary nutrient source, animal serum, ions, elements, calcium, glutamate, magnesium, zinc, iron, potassium, sodium, amino acids, vitamins, glucose, growth factors, hormones, tissue extracts, proteins, small molecules, or any combination thereof.

Non-limiting examples of amino acids that can used in the enrichment media include essential amino acids, phenylalanine, valine, threonine, tryptophan, isoleucine, methionine, leucine, lysine, and histidine, arginine, cysteine, glycine, glutamine, proline, serine, tyrosine, alanine, asparagine, aspartic acid, glutamic acid, or any combination thereof.

Non-limiting examples of growth factors that can be used in the enrichment media include Epidermal Growth Factor (EGF), Nerve Growth Factor (NGF), Brain Derived Neurotrophic Factor (BDNF), Fibroblast Growth Factor (FGF), Stem Cell Factor (SCF), Insulin-like Growth Factor (IGF), Transforming Growth Factor-beta (TGF-β), Basis Fibroblast Growth Factor (bFGF), testosterone, estrogen, thyroid-stimulating hormone (TSH), follicle-stimulating hormone, luteinizing hormone, eicosanoids, melatonin, thyroxine, vasopressin, oxytocin, or any combination thereof.

Non-limiting examples of hormones include peptide hormones, insulin, steroidal hormones, hydrocortisone, progesterone, testosterone, estrogen, dihydrotestosterone, or any combination thereof.

Non-limiting examples of tissue extracts include pituitary extract. Non-limiting examples of small molecule additives include sodium pyruvate, endothelin-1, transferrin, cholesterol, or any combination thereof.

Non-limiting examples of other components that can be used in the enrichment media include pipecolic acid, gamma-Aminobutyric acid (GABA), human serum albumin, bovine serum albumin, glutathione, human alpha-fetoprotein, bovine alpha-fetoprotein, human holo-transferrin, or any combination thereof.

Non-limiting examples of salts that can be used in the enrichment media include calcium chloride, magnesium chloride, sodium bicarbonate, magnesium sulfate, sodium chloride, citrate, potassium phosphate, sodium phosphate, or any combination thereof.

In some embodiments, the enrichment media contains pipecolic acid, GABA, bFGF, TGFβ-1, human insulin, human holo-transferrin, human serum albumin, and reduced glutathione.

The amino acids, growth factors, hormones, tissue extracts, salts, or any other component that can be used in the enrichment media can be at a concentration of, for example, about 0.001 nM, about 0.005 nM, about 0.01 nM, about 0.015 nM, about 0.02 nM, about 0.25 nM, about 0.03 nM, about 0.035 nM, about 0.04 nM, about 0.045 nM, about 0.05 nM, about 0.055 nM, about 0.06 nM, about 0.065 nM, about 0.07 nM, about 0.075 nM, about 0.08 nM, about 0.085 nM, about 0.09 nM, about 0.1 nM, about 0.015 nM, about 0.2 nM, about 0.25 nM, about 0.3 nM, about 0.35 nM, about 0.4 nM, about 0.45 nM, about 0.5 nM, about 0.55 nM, about 0.6 nM, about 0.65 nM, about 0.7 nM, about 0.75 nM, about 0.8 nM, about 0.85 nM, about 0.9 nM, about 0.95 nM, about 0.001 μM, about 0.005 μM, about 0.01 μM, about 0.015 μM, about 0.02 μM, about 0.025 μM, about 0.03 μM, about 0.035 μM, about 0.04 μM, about 0.045 μM, about 0.05 μM, about 0.055 μM, about 0.06 μM, about 0.065 μM, about 0.07 μM, about 0.075 μM, about 0.08 μM, about 0.085 μM, about 0.09 μM, about 0.085 μM, about 0.09 μM, about 0.085 μM, about 0.09 μM, about 0.085 μM, about 0.09 μM, about 0.085 μM, about 0.09 μM, about 0.085 μM, about 0.09 μM, about 0.085 μM, about 0.09 μM, about 0.095 μM, about 0.1 μM, about 0.15 μM, about 0.2 μM, about 0.25 μM, about 0.3 μM, about 0.35 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 0.95 μM, about 0.001 mM, about 0.005 nM, about 0.01 mM, about 0.015 mM, about 0.02 mM, about 0.025 mM, about 0.03 mM, about 0.035 mM, about 0.04 mM, about 0.045 mM, about 0.05 mM, about 0.055 mM, about 0.06 mM, about 0.065 mM, about 0.07 mM, about 0.075 mM, about 0.08 mM, about 0.085 mM, about 0.09 mM, about 0.095 mM, about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 350 mM, about 400 mM, about 450 mM, about 500 mM, about 600 mM, about 700 mM, about 800 nM, about 900 mM, and about 1 M.

The culturing conditions in a method of the invention can be adjusted to simulate oxygen and pressure levels found in a particular microenvironment to promote the collection of a desired cell population. The microenvironment can be, for example, a tumor microenvironment, bone metastatic environment, vasculature environment, or brain microenvironment. The oxygen level used during culturing conditions or in a cell incubator can be, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% oxygen in the incubator. In some embodiments, the cells can be grown under hypoxic conditions.

The culturing condition in a method of the invention can be adjusted to simulate the pressure found in the tumor microenvironment to promote the collection of, for example, CTCs, maintenance of a tumor biopsy, or expansion of a tumor biopsy. The pressure used during culturing conditions can be a PSI gauge (PSIG) reading of, for example, about 0.5 PSIG, about 0.6 PSIG, about 0.7 PSIG, about 0.8 PSIG, about 0.9 PSIG, about 1 PSIG, about 1.1 PSIG, about 1.2 PSIG, about 1.3 PSIG, about 1.4 PSIG, about 1.5 PSIG, about 1.6 PSIG, about 1.7 PSIG, about 1.8 PSIG, about 1.9 PSIG, about 2 PSIG, about 2.5 PSIG, about 3 PSIG, about 3.5 PSIG, about 4 PSIG, about 4.5 PSIG, about 5 PSIG, about 6 PSIG, about 7 PSIG, about 8 PSIG, about 9 PSIG, about 10 PSIG, about 15 PSIG, about 20 PSIG, about 25 PSIG, about 30 PSIG, about 35 PSIG, about 40 PSIG, about 45 PSIG, about 50 PSIG, or about 55 PSIG.

The pressure used during culturing conditions can be, for example, about 3.45 kPa, about 4.14 kPa, about 4.83 kPa, about 5.52 kPa, about 6.21 kPa, about 6.89 kPa, about 7.58 kPa, about 8.27 kPa, about 8.96 kPa, about 9.65 kPa, about 10.3 kPa, about 11 kPa, about 11.7 kPa, about 12.4 kPa, about 13.1 kPa, about 13.8 kPa, about 17.2 kPa, about 20.7 kPa, about 24.1 kPa, about 27.6 kPa, about 31 kPa, about 34.4 kPa, about 41.4 kPa, about 48.3 kPa, about 55.2 kPa, about 62.1 kPa, about 68.9 kPa, about 103 kPa, about 138 kPa, about 172 kPa, about 207 kPa, about 241 kPa, about 276 kPa, about 310 kPa, about 345 kPa, or about 379 kPa.

The pH of the enrichment media used in a method of the invention can be, for example, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.55, about 4.6, about 4.7, about 4.8, about 4.9, about 5, about 5.5, about 6, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.5, about 9, about 9.5, about 10, about 10.5, or about 11 pH units.

The viscosity of the enrichment media can be adjusted by, for example, at least 0.001 Pascal-second (Pa·s), at least 0.001 Pa·s, at least 0.0009 Pa·s, at least 0.0008 Pa·s, at least 0.0007 Pa·s, at least 0.0006 Pa·s, at least 0.0005 Pa·s, at least 0.0004 Pa·s, at least 0.0003 Pa·s, at least 0.0002 Pa·s, at least 0.0001 Pa·s, at least 0.00005 Pa·s, or at least 0.00001 Pa·s depending on the cell types being cultured.

The oxygen solubility of the enrichment media can be, for example, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99%.

A method of the invention can further comprise coating surfaces for cell adhesion with particular media compositions to promote cellular and cellular protein binding to the surface. The surface can be, for example, a cell culture plate, a cell culture plate with multiple wells, a petri dish, a glass slide, a cover slip, or a glass dish. The media used for coating of the cell adhesion surfaces can include, for example, 3-(Aminopropyl)trimethoxysilane, (3-Mercaptopropyl)trimethoxysilane, (3-Aminopropyl)triethoxysilane, N-[3-(Trimethoxysilyl)propyl]ethylenediamine, (3-Glycidyloxypropyl)trimethoxysilane, [3-(2-Aminoethylamino)propyl]trimethoxysilane, Trimethoxy[3-(methylamino)propyl]silane, 3-Aminopropyl(diethoxy)methylsilane, or glutaraldehyde.

The surface coating can further comprise an extracellular matrix (ECM) mix to facilitate cell binding. The mix can include, for example, collagens, basement membrane proteins, collagen IV, laminins, fibronectin, vitronectin, vimentin, tumor-derived extracellular matrix proteins, or inert self-assembling peptides systems. The components used can be animal- or human-derived. The ECM mix can be diluted to a pH of about 4 to about 10 using, for example, potassium hydroxide (KOH), L-glycine, DMEM powder, sodium hydroxide (NaOH), or PBS. The ECM mix can be further supplemented with, for example, human plasma or animal-derived serum. The animal-derived serum can be, for example, bovine serum or fetal bovine serum.

The cells can be cultured in enrichment media, or in a cell culture incubator, for about 1 minute, about 2 minutes, about 3 minutes, about 4 minutes, about 5 minutes, about 10 minutes, about 15 minutes, about 20 minutes, about 25 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 18 hours, about 24 hours, about 30 hours, about 36 hours, about 42 hours, about 2 days, about 3 days, about 4 days, about 5 days, about 6 days, about 1 week, about 2 weeks, about 3 weeks, about 4 weeks, about 2 months, about 3 months, about 4 months, about 5 months, about 6 months, about 7 months, about 8 months, about 9 months, about 10 months, about 11 months, about 1 year, about 1.5 years, about 2 years, about 2.5 years, or about 3 years.

Databases containing information regarding genetic mutations that are prevalent in specific types of cancer can be used to compare the genetic profile or biomarker expression of the target subpopulations derived using the present invention to known mutations. Non-limiting examples of databases that can be used for comparison include COSMIC, cBio Portal, Human Gene Mutation Database (HGMD TM), GWAS central, and the Universal Mutation Database.

### Cell Culture Incubator.

The invention further provides a cell culture incubator. The cell culture incubator can comprise an enclosed environmental chamber. The cell culture incubator can be configured to maintain a gas composition of the enclosed environmental chamber, an atmospheric pressure of the enclosed environmental chamber, humidity, carbon dioxide level, oxygen level, and an internal ambient temperature of the enclosed environmental chamber. The cell culture incubator can comprise a control unit, wherein the control unit can be configured to maintain at least one of the gas composition, the atmospheric pressure, humidity, carbon dioxide level, oxygen level, or the internal ambient temperature. The control unit can be operably linked to the enclosed environmental chamber. The control unit can be configured to maintain at least two of the gas compositions, the atmospheric pressure, humidity, carbon dioxide level, oxygen level, and the internal ambient temperature. The control unit can be configured to maintain the gas composition, the atmospheric pressure, humidity, carbon dioxide level, oxygen level, and the internal ambient temperature. The control unit of the cell culture incubator can be user-controlled or automated based on sensors in the cell culture incubator. The control unit can be configured to create a dynamic gas composition, atmospheric pressure, humidity, carbon dioxide level, oxygen level and the internal ambient temperature as a function of time. The control unit can be configured to cycle between several different gas compositions, atmospheric pressure, humidity level, carbon dioxide level, oxygen level, and the internal ambient temperature as a function of time, and can be stochastic or periodic.

At least one of the gas composition, the atmospheric pressure, humidity, carbon dioxide level, oxygen level, and the internal ambient temperature can be configured for selective proliferation of a target primary cell subpopulation as compared to a non-target primary cell subpopulation. The selective proliferation of a target cell subpopulation can be evidenced by, for example, a two-fold increase in the proliferation rate of the target primary cell subpopulation as compared to the proliferation rate of the non-target primary cell subpopulation. At least one of the gas composition, the atmospheric pressure, humidity, carbon dioxide level, oxygen level, and the internal ambient temperature can be configured for selective adherence of a target primary cell subpopulation as compared to a non-target primary cell subpopulation. The selective adherence of a target primary cell subpopulation can be evidenced by a two-fold increase in adherence of the target primary cell subpopulation as compared to adherence of the non-target primary cell subpopulation. At least one of the gas composition, the atmospheric pressure, humidity, carbon dioxide level, oxygen level, and the internal ambient temperature can be configured to promote selective colony formation of the target primary cell as compared to colony formation of the non-target primary cell subpopulation. The selective colony formation can be evidenced by a two-fold increase in colony formation of the target primary cell subpopulation as compared to colony formation of the non-target primary cell subpopulation. The colony formation can be a two-dimensional or three-dimensional colony formation.

The gas composition in the cell culture incubate can comprise an oxygen level between about 0.1 to about 21%. In some embodiments, the cell culture incubator maintains an oxygen level of the enclosed environmental chamber of no more than about 5%. In some embodiments, the cell culture incubator maintains an oxygen level of the enclosed environmental chamber of no more than about 2%. In some embodiments, the cell culture incubator maintains an oxygen level of the enclosed environmental chamber of no more than about 1%.

The cell culture incubator can maintain a user-controlled or automated atmospheric pressure of the enclosed environmental chamber of about 1 PSIG (6.89 kPa) or greater. In some embodiments, the cell culture incubator maintains a user-controlled atmospheric pressure of the enclosed environmental chamber of about 2 PSIG (13.8 kPa) or greater. In some embodiments, the cell culture incubator maintains a user-controlled atmospheric pressure of the enclosed environmental chamber of about 5 PSIG (34.5 kPa). The cell culture incubator can maintain the atmospheric pressure of the enclosed environmental chamber by controlling an inlet gas pressure.

The cell culture incubator can comprise a user interface. The user interface can be configured to allow a user to control the gas composition, oxygen level, carbon dioxide level, humidity, atmospheric pressure, or internal ambient temperature. The user interface can be configured to provide a display of the gas composition to a user. The user interface can be configured to provide a display of the oxygen level, carbon dioxide level, humidity, atmospheric pressure of the enclosed environmental chamber, and internal ambient temperature to a user. The cell culture incubator can be configured to maintain an internal humidity of the enclosed environmental chamber. The enclosed environmental chamber can comprise a shelf, a pressure sensor, an oxygen sensor, a carbon dioxide sensor, a temperature sensor, or an oxygen removal catalyst. The shelf in the enclosed environmental chamber can be made of, for example, stainless steel, silver, gold, or copper. In some embodiments, the shelf of the enclosed environmental chamber is a copper shelf.

The cell culture incubator can be operably linked to a gas tank. The gas tank can comprise a CO₂ tank, a nitrogen gas tank, an oxygen gas tank, a gas tank comprising a defined mixture of one or more gases, or any combination thereof. The cell culture incubator can be operably linked to the gas tank via a pressurized pump or pressure sensor (e.g., pressure gauge). The pressurized pump or pressure sensor can maintain a controlled flow of gas from the gas tank to the enclosed environmental chamber of the cell culture incubator. The controlled flow of gas from the one or more tanks can have a set inlet pressure, the set inlet pressure of the one or more tanks configured to maintain the desired internal gas composition or internal atmospheric pressure of the enclosed environmental chamber. The enclosed environmental chamber can have a vacuum seal on the door of the enclosed environmental chamber. The enclosed environment chamber can be sealed by an inflatable seal.

The incubator can comprise a pressurized door. In some embodiments, the incubator comprises an outer pressurized door and an inner pressurized door. The outer pressurized door and/or inner pressurized door can be, e.g., a double-walled door. The double-walled door can have a vacuum-sealed latch. The outer pressurized door may include an integrated pressure sensor on the door. The incubator can comprise a door entry. The door entry can provide an entrance into an enclosed environmental chamber. Dimensions of the door entry opening can be less than the pressurized door. The door entry can comprise a rubber gasket. The rubber gasket can create a pressurized seal. The cell culture incubator can comprise an integrated pressure sensor. The integrated pressure sensor can be a manifold pressure sensor. The integrated pressure sensor can be a water-based or silicon based pressure sensor. The incubator can comprise a sterilization unit. The sterilization unit can be a UV-based sterilization unit. The UV-based sterilization unit can be configured to provide UV rays to the entire space of an enclosed environmental chamber of the incubator. The incubator can comprise a CO₂ sensor. The CO₂ sensor can be configured to provide a detectable alarm upon deviation of +/−0.5% from a defined CO₂ level of the enclosed environmental chamber. The incubator can comprise an enclosed environmental chamber. The incubator can comprise a water humidity tray. The water humidity tray can promote sterility of the enclosed environmental chamber. The water humidity tray may be tethered directly to humidity sensor or regulator. The incubator can comprise an air jacket. The air jacket can maintain optimal temperature and proper gas regulation. The air jacket can be a physically distinct compartment. The air jacket may house electrical controllers and circuit boards. The incubator can comprise an oxygen removal catalyst and sensor for regulating oxygen levels within the incubator.

The incubator can comprise a heating element or temperature control. The heating element or temperature control can comprise silent fan-based heating elements. The silent fan-based heating elements can dispense a constant flow of heated air into the air-jacket compartment. Passive heating can then warm the inner chamber in an evenly distributed and constant manner. The incubator can comprise a pressurized pump and regulator configured to provide a defined gas composition and internal atmospheric pressure. A motor based pump can dispense defined gas mixtures to maintain chamber pressure and gas composition levels (e.g., 1% oxygen, 5% CO₂, 94% N₂). The incubator can comprise a user interface. A user can use the user interface to set gas levels and pressures. The user interface can be integrated to the sensor and pump for direct control. The pressurized pump and regulator can comprise a gas inlet. The gas inlet can allow flow of any gas into the enclosed environmental chamber. For example, the gas inlet can be connected to an oxygen tank. The gas inlet can be connected to a CO₂ tank. The gas inlet can be connected to a nitrogen tank. In some embodiments, the incubator comprises a gas inlet connected to an oxygen tank, a gas inlet connected to a CO₂ tank, and a gas inlet connected to a nitrogen tank. The gas inlet can be connected to a tank containing a custom gas mixture. In some embodiments, a user can control a flow of gas through any gas inlet. Any one of the gas inlets may be connected to a flow meter. The flow meter can regulate an inlet gas pressure. The cell culture incubator can comprise a humidity control unit/sensor. The humidity control unit/sensor can be directly connected to the water humidity tray.

The enclosed environmental chamber of the cell culture incubator can comprise a sterilization unit, which can be a UV sterilization unit. The enclosed environmental chamber can comprise a pressurized door. The enclosed environmental chamber can comprise a sensor that provides a detectable alarm upon detection of an oxygen level of the enclosed environmental chamber that differs by more than about ±0.5% from a user-desired oxygen level. The enclosed environmental chamber can comprise a sensor that provides a detectable alarm upon detection of an atmospheric pressure of the enclosed environmental chamber that differs by more than about ±0.5% from a user-desired atmospheric pressure. The enclosed environmental chamber can comprise a user display that displays an atmospheric pressure level of the enclosed environmental chamber. In some embodiments, the enclosed environmental chamber comprises a user display that displays an O₂ level of the enclosed environmental chamber. In some embodiments, the enclosed environmental chamber comprises a user display which displays a CO₂ level of the enclosed environmental chamber. In some embodiments, the enclosed environmental chamber comprises a user display which displays a temperature level of the enclosed environmental chamber.

A user can program the cell culture incubator to mimic, for example, physiological, tumor microenvironment, hypoxic, high pressure, low pressure, or supraphysiological conditions. The cell culture incubator can be configured to calibrate to the conditions set by the user within about one minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 30 minutes, about 35 minutes, about 40 minutes, about 45 minutes, about 50 minutes, about 55 minutes, or about one hour. In some embodiments, the cell culture incubator can reach the desired conditions set by the user in less than about 20 minutes. In some embodiments, the cell culture incubator can reach the desired conditions set by the user in about 20 minutes. In some embodiments, the cell culture incubator can reach the desired conditions set by the user within 20 minutes.

In some embodiments, the enclosed environmental chamber occupies no more than 6 cubic feet of space. In some embodiments, the enclosed environmental chamber occupies no more than 3.5 cubic feet of space. In some embodiments, the enclosed environmental chamber occupies no more than 2 cubic feet of space. In some embodiments, the enclosed environmental chamber occupies no more than 1.5 cubic feet of space. In some embodiments, the enclosed environmental chamber occupies no more than 1 cubic foot of space. In some embodiments, the enclosed environmental chamber occupies less than 1 cubic foot of space.

In some embodiments, the cell culture plate comprises 1, 6, 12, 24, 48, 96, 384, 1056, 1536, or 3456 wells.

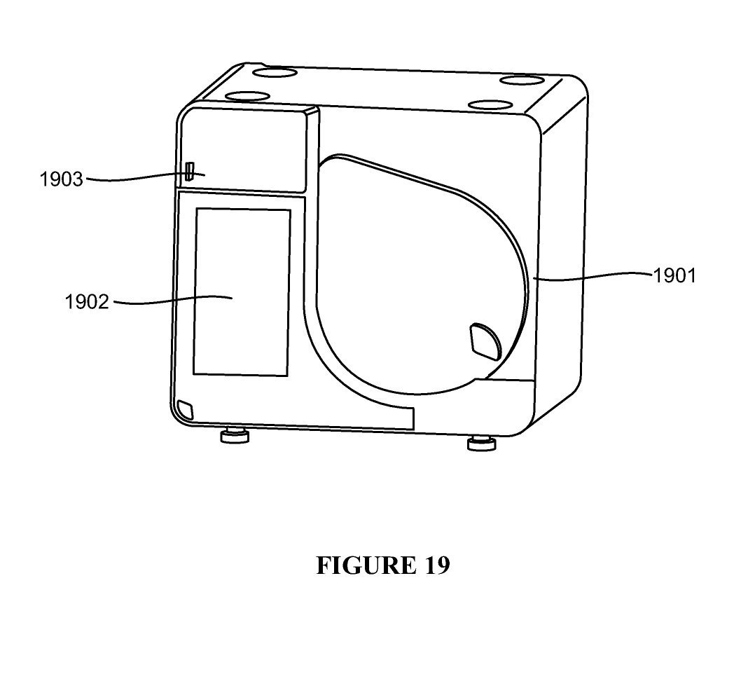

A method of the invention can employ a cell culture incubator for culturing of a target cell population. FIG. 19 provides an illustrative example of a cell culture incubator that can be used in a system of the invention. 1901 is the door of the incubator that can be opened to place a cell culture in the incubator. 1902 is a control unit that can be used to program the cell culture incubator using parameters including, for example, temperature, humidity, oxygen level, carbon dioxide level, time of incubation, nitrogen level, and chamber pressure. 1903 is a USB port that can be used to input data to or extract data from the cell culture incubator.

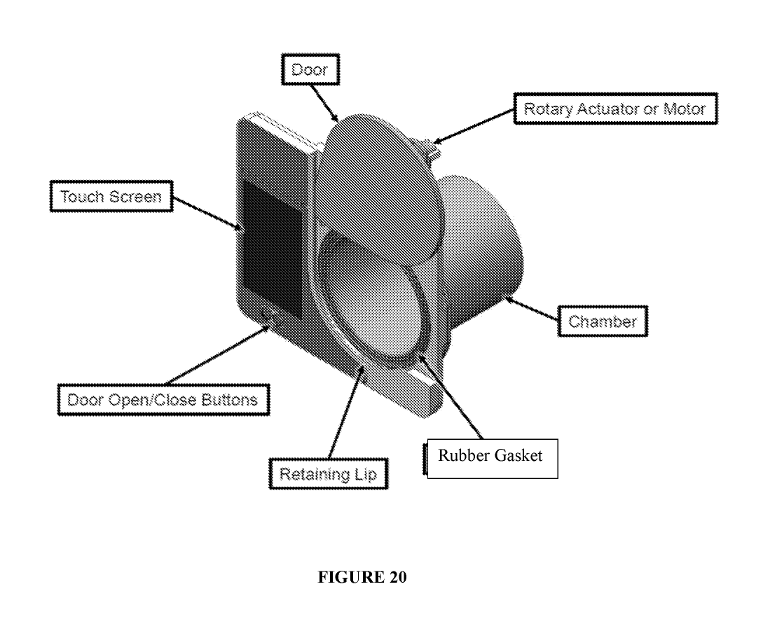

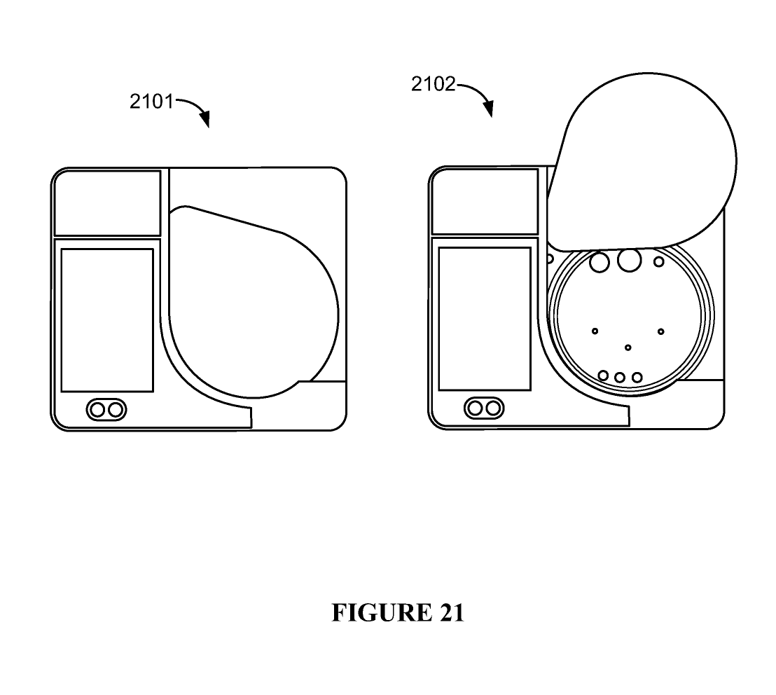

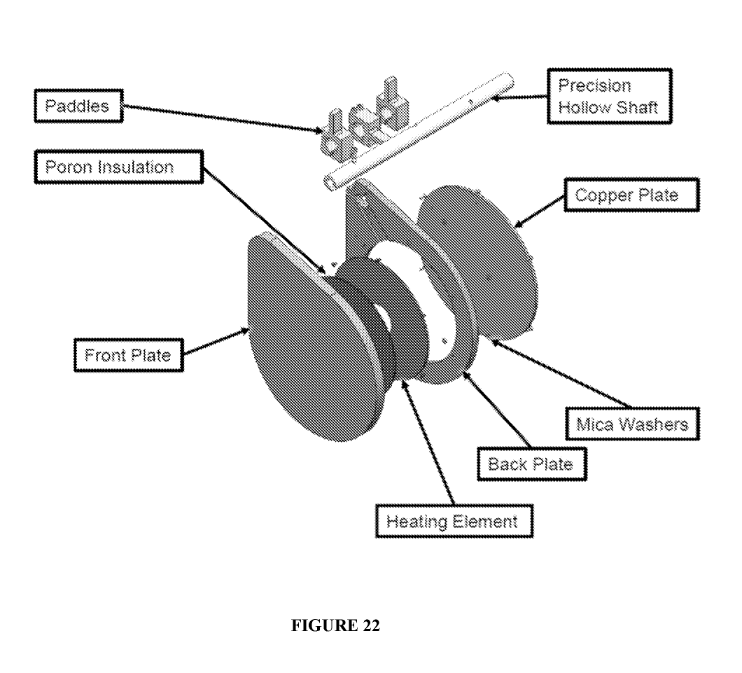

FIG. 20 is a diagram of the front of the cell culture incubator. FIG. 20 depicts the door, a rubber gasket around the edge of the door that can fit tightly using a door handle latch, retaining lip, open and close buttons for the door, touch screen, rotary actuator or motor, and chamber of the incubator. FIG. 21 shows the cell culture incubator door closed (2101) and open (2102). FIG. 22 depicts a door heater than can be used in a system of the invention. The figure shows the front plate, heating element, poron insulation, back plate, mica washers, copper plate, precision hollow shaft, and paddles that can be used to heat the cell culture incubator to a desired temperature.

The height, width, depth, or length of the cell culture incubator can be, for example, about 6 in, about 6.5 in, about 7 in, about 7.5 in, about 8 in, about 8.5 in, about 9 in, about 9.5 in, about 10 in, about 10.5 in, about 11 in, about 11.5 in, about 12 in, about 12.1 in, about 12.2 in, about 12.3 in, about 12.4 in, about 12.5 in, about 12.6 in, about 12.7 in, about 12.8 in, about 12.9 in, about 13 in, about 13.1 in, about 13.2 in, about 13.3 in, about 13.4 in, about 13.5 in, about 13.6 in, about 13.7 in, about 13.8 in, about 13.9 in, about 14 in, about 14.5 in, about 15 in, about 15.5 in, about 16 in, about 16.5 in, about 17 in, about 17.5 in, about 18 in, about 18.5 in, about 19 in, about 19.5 in, about 20 in, about 20.5 in, about 21 in, about 21.5 in, about 22 in, about 22.5 in, about 23 in, about 23.5 in, about 24 in, about 24.5 in, about 25 in, about 25.5 in, about 26 in, about 26.5 in, about 27 in, about 27.5 in, about 28 in, about 28.5 in, about 29 in, about 29.5 in, about 30 in, about 30.5 in, about 31 in, about 31.5 in, about 32 in, about 32.5 in, about 33 in, about 33.5 in, about 34 in, about 34.5 in, about 35 in, about 35.5 in, about 36 in, about 36.5 in, about 37 in, about 37.5 in, about 38 in, about 38.5 in, about 39 in, about 39.5 in, about 40 in, about 40.5 in, about 41 in, about 41.5 in, about 42 in, about 42.5 in, about 43 in, about 43.5 in, about 44 in, about 44.5 in, about 45 in, about 45.5 in, about 46 in, about 46.5 in, about 47 in, about 47.5 in, about 48 in, about 48.5 in, about 49 in, about 49.5 in, about 50 in, about 50.5 in, about 51 in, about 51.5 in, about 52 in, about 52.5 in, about 53 in, about 53.5 in, about 54 in, about 54.5 in, about 55 in, about 55.5 in, about 56 in, about 56.5 in, about 57 in, about 57.5 in, about 58 in, about 58.5 in, about 59 in, about 59.5 in, about 60 in, or any combination thereof.

In some embodiments, the height of the cell culture incubator is 12 in. In some embodiments, the width of the cell culture incubator is 13.5 in. In some embodiments, the depth of the cell culture incubator is 13.1 in.

The capacity of the enclosed environmental chamber can be, for example, about 100 inch³, about 110 inch³, about 120 inch³, about 130 inch³, about 140 inch³, about 150 inch³, about 160 inch³, about 170 inch³, about 180 inch³, about 190 inch³, about 200 inch³, about 205 inch³, about 210 inch³, about 211 inch³, about 212 inch³, about 213 inch³, about 214 inch³, about 215 inch³, about 216 inch³, about 217 inch³, about 218 inch³, about 219 inch³, about 220 inch³, about 221 inch³, about 222 inch³, about 223 inch³, about 224 inch³, about 225 inch³, about 226 inch³, about 227 inch³, about 228 inch³, about 229 inch³, about 230 inch³, about 240 inch³, about 250 inch³, about 260 inch³, about 270 inch³, about 280 inch³, about 290 inch³, about 300 inch³, about 310 inch³, about 320 inch³, about 330 inch³, about 340 inch³, about 340 inch³, about 350 inch³, about 360 inch³, about 370 inch³, about 380 inch³, about 390 inch³, about 400 inch³, about 420 inch³, about 440 inch³, about 460 inch³, about 480 inch³, or about 500 inch³. In some embodiments, the capacity of the enclosed environmental chamber is about 220 inch³. In some embodiments, the capacity of the enclosed environmental chamber is about 221 inch³. In some embodiments, the capacity of the enclosed environmental chamber is about 222 inch³. In some embodiments, the capacity of the enclosed environmental chamber is about 223 inch³. In some embodiments, the capacity of the enclosed environmental chamber is about 224 inch³. In some embodiments, the capacity of the enclosed environmental chamber is 224 inch³.

Materials that can be used in the manufacture of the cell culture incubator include, for example, stainless steel, glass, copper, silver, gold, plastic, blanket batting, hard-board insulation, or any combination thereof. The enclosed environmental chamber can be made of, for example, copper or stainless steel. In some embodiments, the enclosed environmental chamber is made of copper.

The cell culture incubator can be maintained at a desired humidity level. The humidity level can be, for example, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.5%, or about 99.9%.

The CO₂ levels in the cell culture incubator can be, for example, about 10%, about 9.5%, about 9%, about 8.5%, about 8%, about 7.5%, about 7%, about 6.9%, about 6.8%, about 6.7%, about 6.6%, about 6.5%, about 6.4%, about 6.3%, about 6.2%, about 6.1%, about 6%, about 5.9%, about 5.8%, about 5.7%, about 5.6%, about 5.5%, about 5.4%, about 5.3%, about 5.2%, about 5.1%, about 5%, about 4.9%, about 4.8%, about 4.7%, about 4.6%, about 4.5%, about 4.4%, about 4.3%, about 4.2%, about 4.1%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1%.

The cell culture incubator can be used in combination with the culturing conditions described herein, for example, to isolate specific cell populations, to induce changes in cells, to introduce exogenous materials into cells, to determine biomarker expression, and as a diagnostic tool for patients.

The methods of the invention can be used to increase, for example, transfection and transduction efficiency in cells. Transduction can be used, for example, to introduce a viral vector in a cell. Viral nucleic acid delivery systems can use recombinant viruses to deliver nucleic acids for gene therapy. Non-limiting examples of viruses that can be used to deliver nucleic acids include retrovirus, adenovirus, herpes simplex virus, adeno-associated virus, vesicular stomatitis virus, reovirus, vaccinia, pox virus, and measles virus.

Transfection methods that can be used with methods of the invention include, for example, lipofection, electroporation, calcium phosphate transfection, chemical transfection, polymer transfection, gene gun, magnetofection, or sonoporation. The transfection can be a stable or transient transfection. The transfection can be used to transfect DNA plasmids, RNA, siRNA, shRNA, or any nucleic acid. The plasmids can encode, for example, green fluorescent protein (GFP), selectable markers, and other proteins of interest. The selectable markers can provide resistance to, for example, G418, hygromycin B, puromycin, and blasticidin. The transfection method used with a method of the invention can further introduce a vector system encoding the CRISPR-Cas9 system into a cell.

A method of the invention can increase the transfection or transduction efficiency by, for example, about 2-fold, about 3-fold, about 4-fold, about 5-fold, about 6-fold, about 7-fold, about 8-fold, about 9-fold, about 10-fold, about 12-fold, about 14-fold, about 16-fold, about 18-fold, about 20-fold, about 25-fold, about 30-fold, about 35-fold, about 40-fold, about 45-fold, about 50-fold, about 60-fold, about 70-fold, about 80-fold, about 90-fold, or about 100-fold.

### Therapeutic Uses.

Subjects can be, for example, elderly adults, adults, adolescents, pre-adolescents, children, toddlers, infants. Subjects can be non-human animals, for example, a subject can be a mouse, rat, cow, horse, donkey, pig, sheep, dog, cat, or goat. A subject can be a patient.

A method of the invention can be used to treat or diagnose, for example, cancer in a subject. A method of the invention can be used to identify a therapeutic, a biomarker, a genetic mutation, an epigenetic marker, or a therapeutic target for cancer. A method of the invention can also be used to develop a library or database of genetic mutations found in cancer. A method of the invention can be used for personalized medicine. A method of the invention can be used to determine the effect of a therapeutic on a specific cell type.

A method of the invention can be used, for example, to enrich specific populations of cells or induce expression of specific genes, for example, biomarkers or epigenetic markers. A method of the invention can be used, for example, to affect the potency of stem cells or somatic cells. For example, a method of the invention can be used to test the ability of stem cells to go from, for example, totipotent to, for example, pluripotent, oligopotent, or unipotent.

The change in gene expression can affect, for example, cell quantity, cell morphology, cell growth, cell motility, cell invasion, or cell adhesion.

Genomic, proteomic, and metabolic analysis can be conducted on the cultured cells to, for example, identify biomarkers that can be used for development of cancer therapies, drug development, cancer vaccines, cancer screening, diagnostics, personalized antibody development, hematopoietic stem cell transplantation, organ transplantation, or cardiovascular disease treatment.

Non-limiting examples of cancers that can be analyzed in a method of the invention include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyo sarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

Cell surface marker molecules, such as EPCAM, CD133, EGFR, HER2, or CD20 can be used to identify a population of cells. In some embodiments, a combination of cell surface markers, or a cell surface marker signature, can be used to identify a population of cells. In some embodiments, a cell surface marker, and/or a cell surface marker signature can be used in medical diagnosis.

Epigenetic markers that can be assessed using a method of the invention include, for example, DNA methylation, cytosine methylation, hydroxymethylation, histone methylation, lysine acetylation, lysine methylation, arginine methylation, serine phosphorylation, threonine phosphorylation, protein phosphorylation, protein ubiquitination, protein sumoylation, presence of 5-methylcytosine, histone H3 acetylation, or histone H4 acetylation.

Methods that can be used to determine the presence of biological markers include, for example, qPCR, RT-PCR, immunofluorescence, immunohistochemistry, western blotting, high-throughput sequencing, ELISA, or mRNA sequencing.

Target cell subpopulations can be used for personalized medicine. For example, CTCs and CSCs can be used for chemosensitivity testing whereby chemotherapy regimens can be tested on cultured CTCs. An assessment of the effects of chemotherapy drugs on CTCs including, for example, cell viability and cell division, can be done to determine the efficacy of a given drug.

The methods of the present invention can be used to monitor subject response to a given cancer therapy conducted by serial monitoring of the subject's CTC population as treatment progresses. Blood samples can be analyzed on a regular basis, before, during, and after treatment to assess CTC viability.

The methods of the invention can be used to monitor subjects who are currently in remission to investigate the potential of cancer relapse. Serial testing of subject blood for CTCs can be conducted on a regular basis to determine the potential or likelihood for cancer relapse. In some cases, serial testing can result in earlier detection of relapse. Serial testing can also be used for long-term longitudinal studies.

A method of the invention can be used to collect data about patients for patient stratification during clinical trials. For example, the presence of a specific biomarker found in a patient's CTCs can be used to place the patient in appropriate clinical trial groups, or can be used as exclusion criteria for other clinical trial groups.

The invention described herein can provide data that can be used for a medical professional to treat a patient. Treatment of a patient can include diagnosis, prognosis or theranosis. Diagnoses can comprise determining the condition of a patient. Diagnosis can be conducted at one time point or on an ongoing basis. For example, a patient can be diagnosed with cancer. In another example, a cancer patient who is in remission can be routinely screened to determine if a cancer relapse has occurred. Prognosis can comprise determining the outcome of a patient's disease, the chance of recovery, or how the disease will progress. For example, identifying CTCs of a certain type can provide information upon which a prognosis can be based. Theranosis can comprise determining a therapy treatment. For example, a patient's cancer therapy treatment can include chemotherapy, radiation, drug treatment, no treatment, or any combination thereof. A patient can be monitored, for example by serial blood testing, to measure CTC populations before, during and after a patient undergoes treatment. A positive response to therapy can result in a decreased CTC viability and lower division rates.

### Computer Systems.

A method of the invention can be used to, for example, sequence, image, or characterize the collected target cell subpopulations. Further methods can be found in PCT/US14/13048, the entirety of which is incorporated herein by reference.

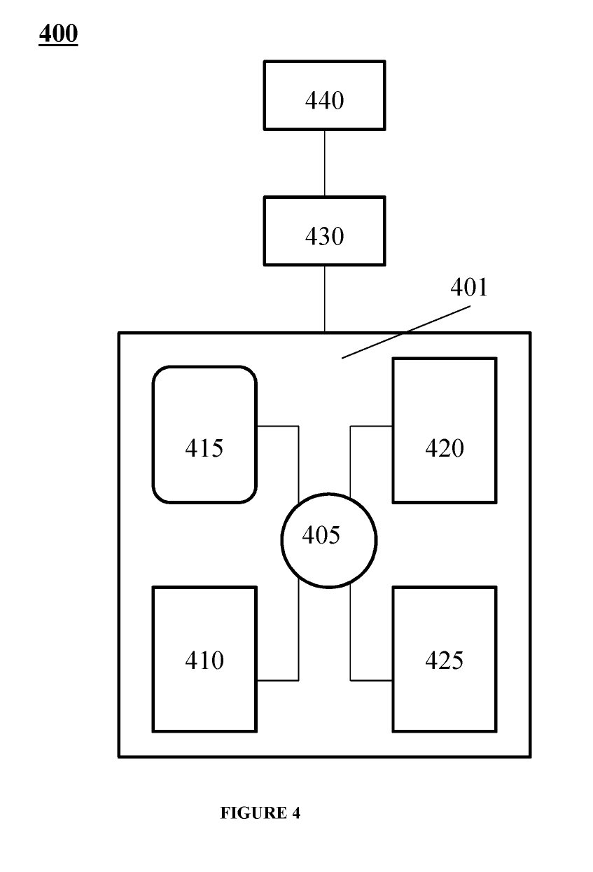

The invention provides a computer system that is configured to implement the methods of the disclosure. The system can include a computer server (“server”) that is programmed to implement the methods described herein. FIG. 4 depicts a system 400 adapted to enable a user to detect, analyze, and process images of cells and sequence cells. The system 400 includes a central computer server 401 that is programmed to implement exemplary methods described herein. The server 401 includes a central processing unit (CPU, also “processor”) 405 which can be a single core processor, a multi core processor, or plurality of processors for parallel processing. The server 401 also includes memory 410 (e.g. random access memory, read-only memory, flash memory); electronic storage unit 415 (e.g. hard disk); communications interface 420 (e.g. network adaptor) for communicating with one or more other systems; and peripheral devices 425 which may include cache, other memory, data storage, and/or electronic display adaptors. The memory 410, storage unit 415, interface 420, and peripheral devices 425 are in communication with the processor 405 through a communications bus (solid lines), such as a motherboard. The storage unit 415 can be a data storage unit for storing data. The server 401 is operatively coupled to a computer network (“network”) 430 with the aid of the communications interface 420. The network 430 can be the Internet, an intranet and/or an extranet, an intranet and/or extranet that is in communication with the Internet, a telecommunication or data network. The network 430 in some cases, with the aid of the server 401, can implement a peer-to-peer network, which may enable devices coupled to the server 401 to behave as a client or a server. The microscope and micromanipulator can be peripheral devices 425 or remote computer systems 440.

The storage unit 415 can store files, such as individual images, time lapse images, data about individual cells, cell colonies, or any aspect of data associated with the invention. The data storage unit 415 may be coupled with data relating to locations of cells in a virtual grid.

The server can communicate with one or more remote computer systems through the network 430. The one or more remote computer systems may be, for example, personal computers, laptops, tablets, telephones, Smart phones, or personal digital assistants.

In some situations the system 400 includes a single server 401. In other situations, the system includes multiple servers in communication with one another through an intranet, extranet and/or the Internet.

The server 401 can be adapted to store cell profile information, such as, for example, cell size, morphology, shape, migratory ability, proliferative capacity, kinetic properties, and/or other information of potential relevance. Such information can be stored on the storage unit 415 or the server 401 and such data can be transmitted through a network.

Methods as described herein can be implemented by way of machine (e.g., computer processor) computer readable medium (or software) stored on an electronic storage location of the server 401, such as, for example, on the memory 410, or electronic storage unit 415. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410. Alternatively, the code can be executed on a second computer system 440.

Aspects of the systems and methods provided herein, such as the server 401, can be embodied in programming. Various aspects of the technology may be thought of as “products” or “articles of manufacture” typically in the form of machine (or processor) executable code and/or associated data that is carried on or embodied in a type of machine readable medium (e.g., computer readable medium). Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

### EXAMPLES

### Example 1: Identification of Markers Associated with Prostate Cancer

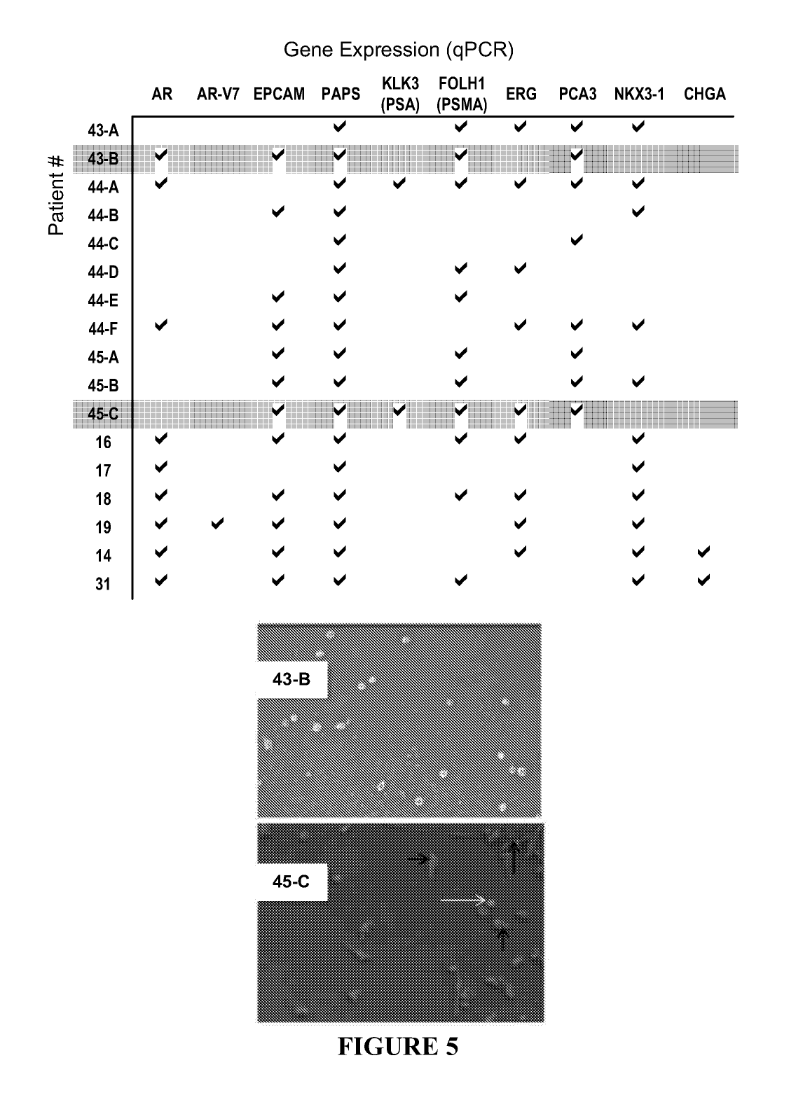

FIG. 5 shows the results of an experiment measuring expression levels of cancer-associated proteins using samples of CTCs obtained from a sample of patients with prostate cancer. The CTCs were grown and cultured according to a method of the invention to obtain an enriched CTC population. Gene expression of various markers associated with prostate cancer was assessed using qPCR. CD45 expression was assessed via immunofluorescence for two subjects, 43-B and 45-C. The white arrow indicates staining for CD45 and the black arrows indicate staining for EPCAM (epithelial cell adhesion molecule)/PSMA. The top panel only contains CD45 staining. The markers assessed included androgen receptor (AR), androgen receptor splice variant 7 (AR-V7), EPCAM, prostatic acid phosphatase (PAPS), prostate-specific antigen (KLK3/PSA), prostate-specific membrane antigen (FOLH1/PSMA), v-ets avian erythroblastosis virus E26 oncogene homolog (ERG), prostate cancer antigen 3 (PCA3), Nk3 homeobox 1 (NKX3-1), and chromogranin A or parathyroid secretory protein 1 (CHGA).

The results indicated that PAPS was expressed in all prostate tumor samples. The expression of other prostate cancer markers differed among the samples indicating that CTC colonies can be genetically diverse between subjects.

### Example 2: Identification of Markers Associated with Prostate Cancer

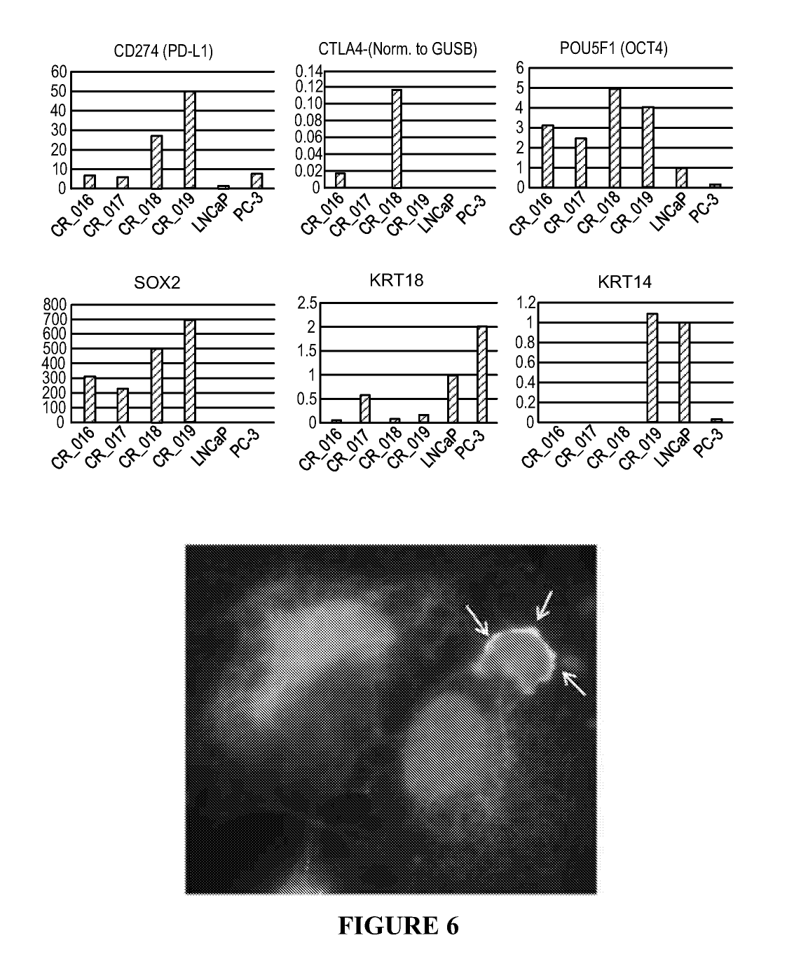

To identify immunotherapeutic targets and stem cell markers that can be expressed by prostate CTC colonies, 10 to 20 mL of peripheral blood was collected from over 30 subjects with metastatic CRPC (mCRPC). Eight of the subject samples yielded CTC colonies after culturing using a method of the invention as described in FIGS. 2-3. Four of the samples were used for qPCR analysis of several markers and were compared to the LNCaP (prostate adenocarcinoma) and PC-3 (prostate adenocarcinoma) cell lines as shows in FIG. 6, which contains the same staining pattern described for FIG. 1.

A representative immunofluorescence image is shown with DAPI, WBC, cytokeratin, and PSMA/PSA staining as in FIG. 1. The results indicated that several of the patient samples and cell lines expressed the immunotherapeutic targets programmed death ligand 1 (CD274/PD-L1) and cytotoxic T-lymphocyte-associated protein 4 (CTLA4) and the stem cell markers octamer-binding transcription factor 4 (PUSF1/Oct4), SRY (sex determining region Y)-box 2 (SOX2), keratin 18, type 1 (KRT18), and keratin 14, type 1 (KRT14), and that there was upregulation in Oct4, SOX2, and PD-L1.

### Example 3: RNA Sequencing of Prostate Cancer CTC Colonies

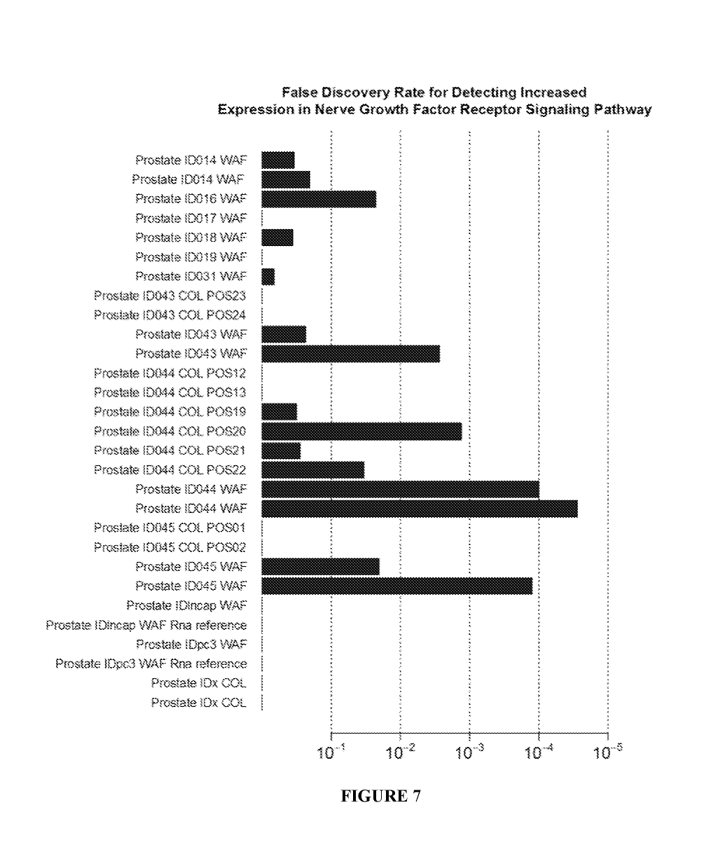

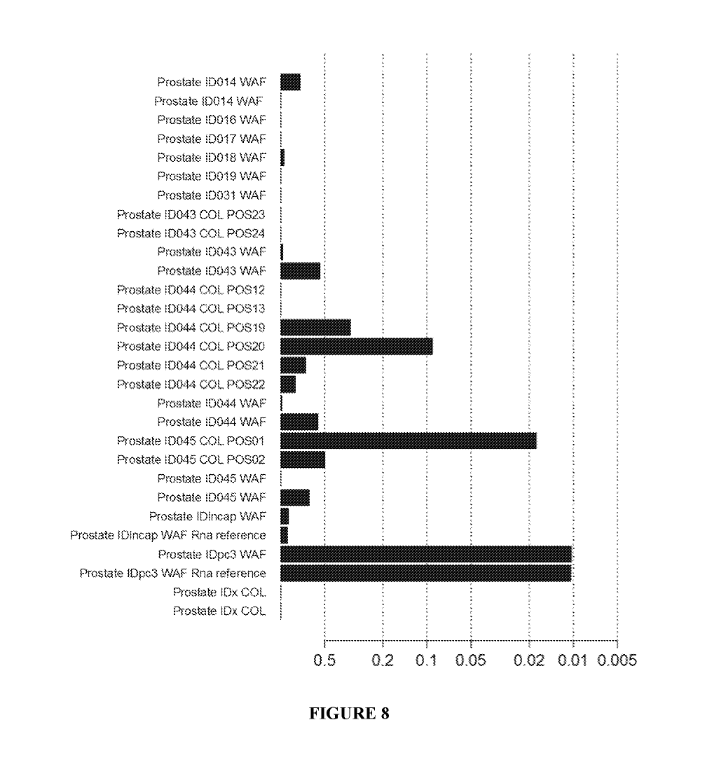

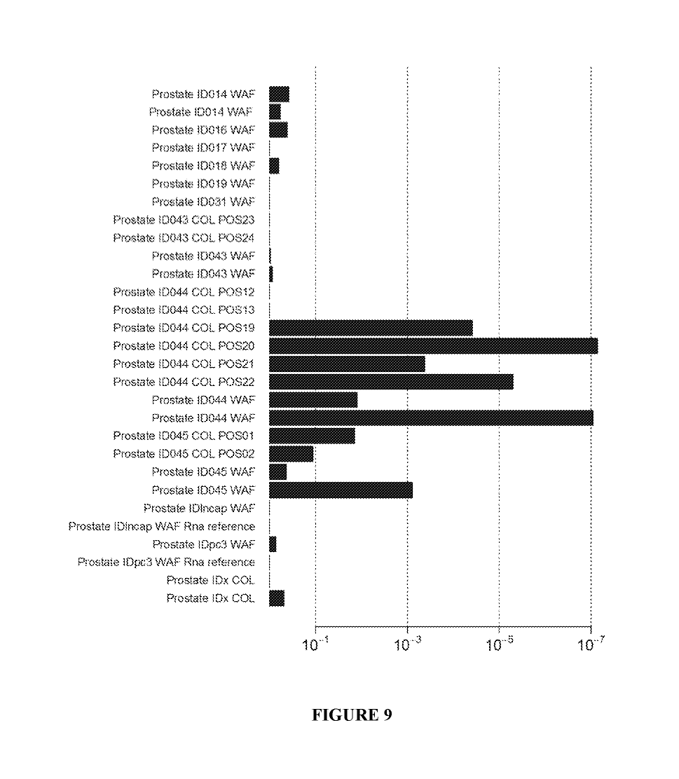

FIGS. 7-9 display results of a mRNA sequencing analysis to determine differential expression of specific signaling pathways among CTC colonies obtained from a sample of subjects. The CTC colonies were obtained using a method as described in FIG. 2-3. The RNA sequence of the cells was mapped to specific genes, and the gene counts were normalized across a collection of samples. Using a non-parametric enrichment algorithm, statistical tests were performed to detect pathways associated with relatively high expression in each sample. False discovery rates were calculated across large collections of pathways. The enrichment test results were expressed as a false discovery rate on the x-axis for each prostate sample RNA profile as seen in FIGS. 7-9. The enrichment for gene expression in different pathways was different across the samples. Pathways that show enrichment in prostate CTC colonies included Nerve Growth Factor Receptor Signaling (FIG. 7), Aurora A Signaling (FIG. 8), and Kit Receptor Signaling (FIG. 9)

### Example 4: EPCAM Expression of Pancreatic CTC Colonies

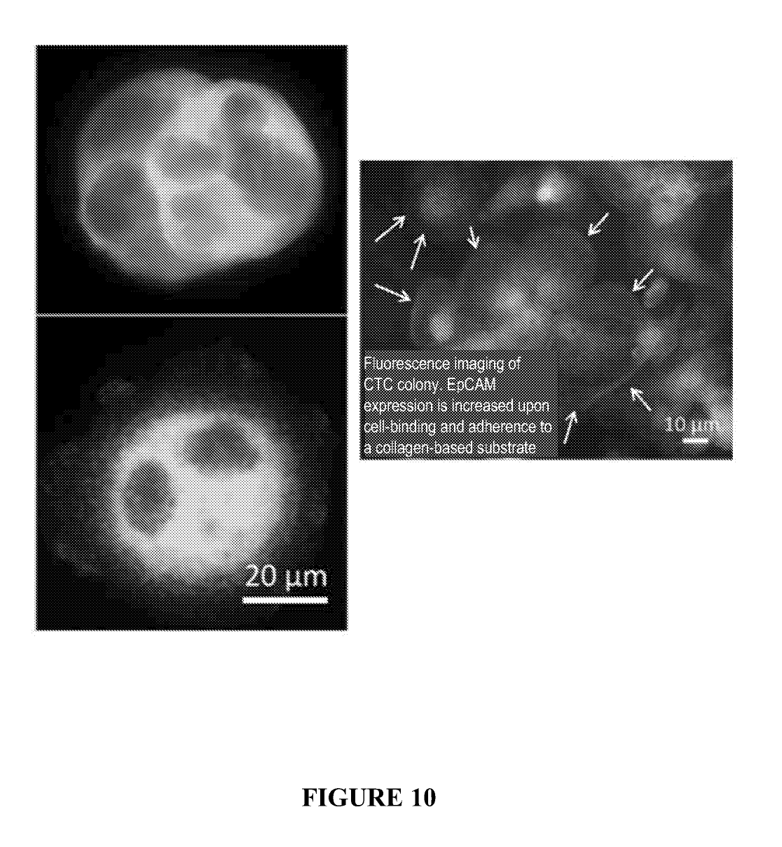

To determine EPCAM expression in pancreatic CTC colonies, 6 patients with pancreatic ductal adenocarcinoma (PDAC) were profiled. Apheresed blood samples were collected and cultured to yield CTC colonies. The cells were stained for cytokeratin 19 (CK19; top left panel, and circular staining in right panel) and EPCAM (punctate staining around cell in bottom left panel, and peripheral staining indicated by white arrows in right panel) as shown in FIG. 10. Cell binding to the collagen-based substrate used for culturing of the CTCs led to increased expression of EPCAM.

### Example 5: Mutations Exhibited by Pancreatic CTC Colonies

Six pancreatic CTC colony samples were analyzed for mutations found in pancreatic cancer as determined from the COSMIC (Catalogue of Somatic Mutations in Cancer) database. In the COSMIC database for PDAC tumors, the mutation rate in KRAS is 69%, p53 is 51%, cyclin-dependent kinase inhibitor 2A (CDKN2A) is 23%, SMAD4 is 21%, AT-rich interactive domain-containing protein 1A (ARID1A) is 6%, and beta-catenin (CTNNB1) is 2%. For CTC colonies obtained after culturing, 2/6 of the colonies displayed mutations in KRAS, and 1/6 colonies displayed mutations in p53, CDK2NA, and CTNNB1.

### Example 6: Gene Expression in Pancreatic and mCRPC CTC Colonies

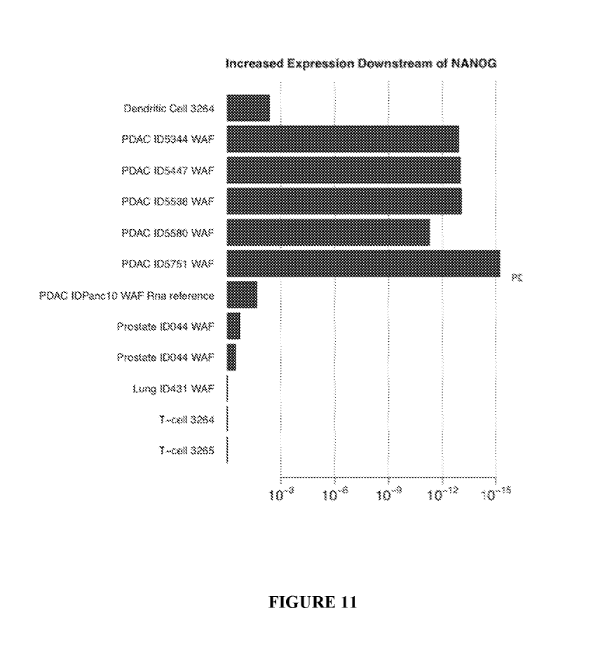

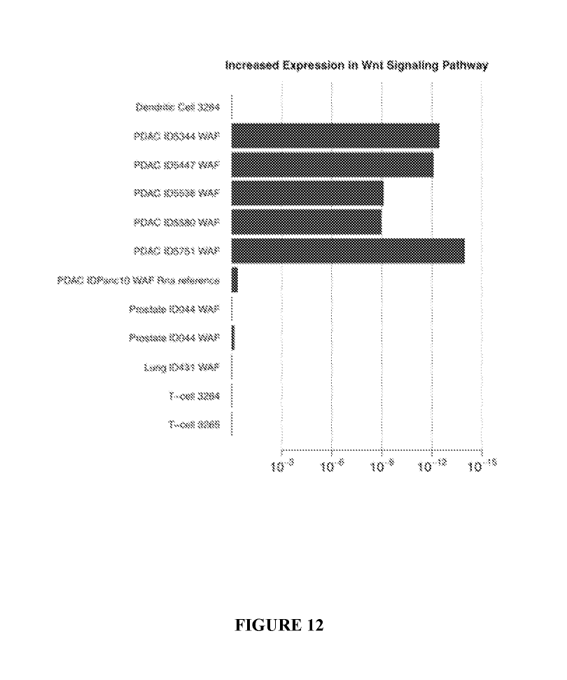

To determine whether there was differential expression between CTCs obtained from different tumors, PDAC CTCs were compared to mCRPC CTCs. Pancreatic CTCs exhibited increased gene expression in the NANOG, Wnt, insulin-like growth factor 1 (IGFR1), FOXP1, and AR signaling pathways. The RNA sequence of the cells was mapped to specific genes, and the gene counts were normalized across a collection of samples. Using a non-parametric enrichment algorithm, statistical tests were performed to detect pathways associated with relatively high expression in each sample. False discovery rates were calculated across large collections of pathways. The enrichment test results were expressed as a false discovery rate on the x-axis for each prostate sample RNA profile as seen in FIGS. 11 and 12. The enrichment for gene expression in different pathways was different across the samples. Pathways that show enrichment in pancreatic CTC colonies over prostate cancer CTCs included the NANOG signaling pathway (FIG. 11) and the Wnt signaling pathway (FIG. 12). Dendritic cells, lung and T-cells were used as controls, and the rest of the samples were CTCs obtained from the subjects.

### Example 7: SNP/INDEL Variant Analysis

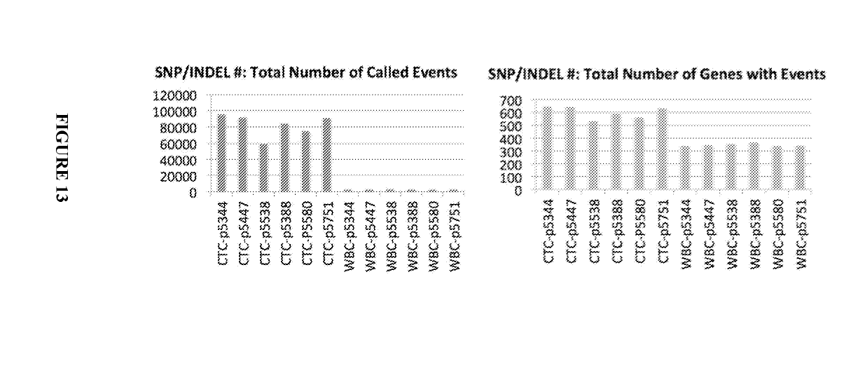

To determine if CTC fractions displayed more genetic variation than whole blood cell controls, single nucleotide polymorphisms (SNPs) and insertions/deletions (INDELs) were analyzed for six patients with stage 4 PDAC. FIG. 13 depicts the results of SNP and INDEL analysis and indicates that both in terms of total events (left panel) and total number of genes with events (right panel), the CTC samples were able to uncover more genetic variants compared to whole blood cell controls.

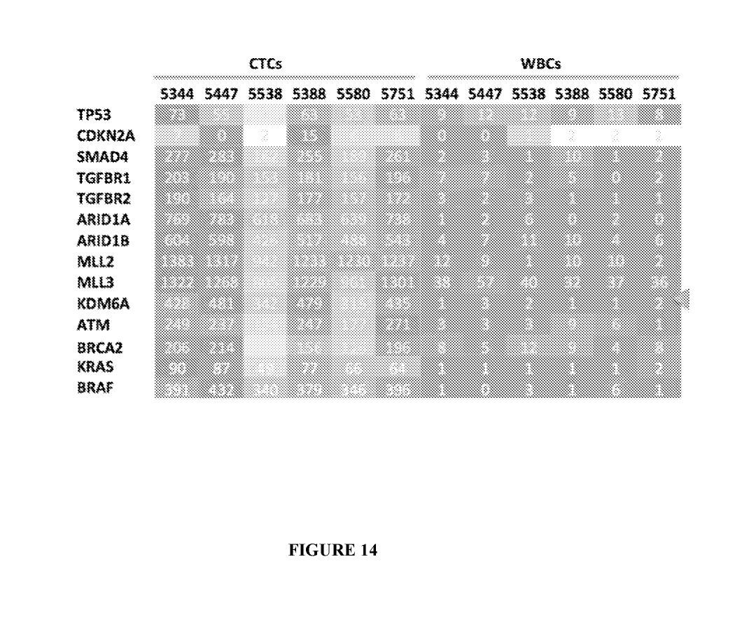

Using a targeted sequencing method, patient samples were assessed for 238 genes associated with PDAC. FIG. 14 illustrates the results of the sequencing analysis and shows that CTCs (left side) revealed about 20-30 times more SNPs and INDELs compared to whole blood cell controls for specific genes. The numbers at the top of the chart denote the patient from which the sample was obtained.

### Example 8: User Interfaces for Displays in a System of the Invention

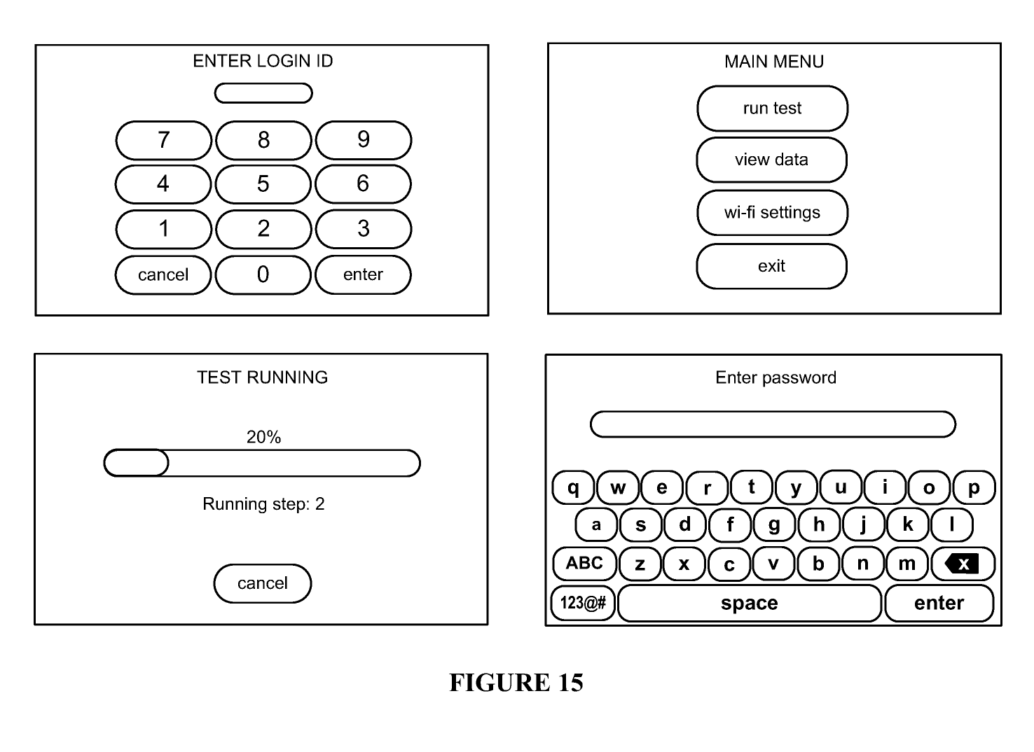

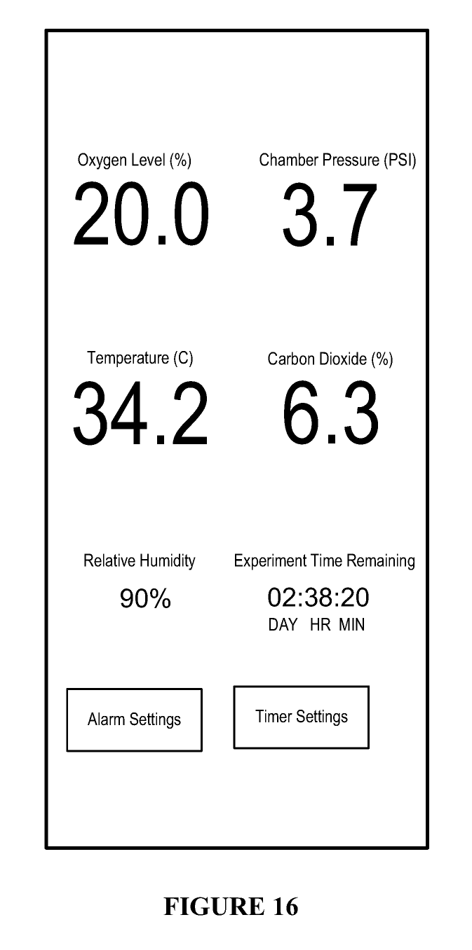

FIG. 15 depicts a graphical interface that is seen by the user upon initialization of a system of the invention. In the top-left corner, the user enters his/her user identification code. In the top-right corner, the user selects the desired option. The quadrant at the bottom-left corner indicates the progress of the desired operation. The bottom-right corner allows the user to enter his/her password for the system. FIG. 16 depicts an additional user interface that can be displayed by a system of the invention upon logging into the system. The user interface of FIG. 16 can be configured to be displayed, for example, for a minimum duration of time, until the hardware connectivity is verified, and until the user hits the start button.

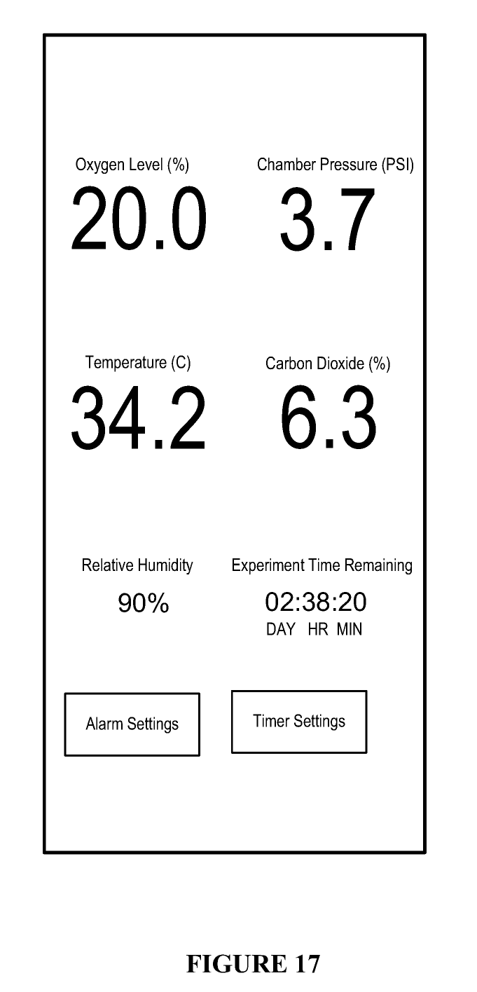

FIG. 17 is a screen displaying the status of the oxygen level (%), chamber pressure (PSI), temperature (° C.), and carbon dioxide level (%) in the culture chamber. The screen further displays the relative humidity and experiment time remaining. The user can further enter alarm settings and time settings using the icons at the bottom of the screen. In this scenario, the oxygen level was 20.0%, the chamber pressure was 3.7 PSIG, the temperature was 34.2° C., and the carbon dioxide level was 6.3%. The relative humidity was 90%, and the experiment time remaining was 2 hours 38 minutes and 20 seconds.

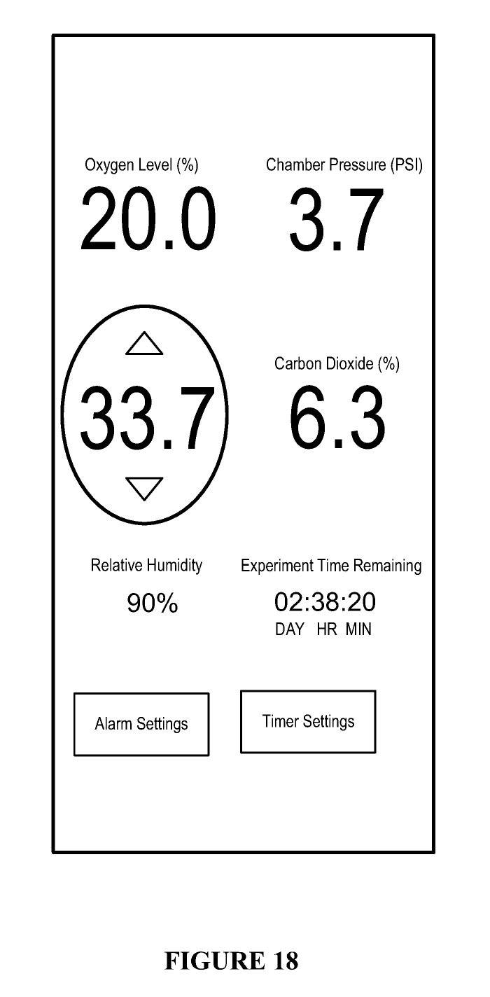

FIG. 18 shows that upon selection of a specific parameter, the user can touch the arrows and decrease or increase the parameter to a desired value. The user can tap the arrow to change the value in small increments, or hold down the arrow to change the value at larger increments. Once the user has reached the desired value, the user touches the value, and the desired value becomes confirmed.

### Example 9: Coating Process for Cell Adhesion

To prepare a surface for covalent binding of cellular proteins, glass slides were prepared by incubating the slides with 0.1 M hydrochloric acid (HCl) for two hours to overnight at room temperature. Then, the glass slides were incubated with 0.1 M NaOH for two hours to overnight at room temperature. The slides were then incubated with 0.5-5% (3-Aminopropyl)trimethoxysilane for two hours to overnight at room temperature. Then, the slides were incubated with 0.5-5% glutaraldehyde (diluted in PBS) for 2 hours to overnight at room temperature. The glass slides were then rinsed with water overnight, sterilized under UV light for one hour, and then stored dry at room temperature.

The slides were then further treated to facilitate cell binding using a mixture containing extracellular matrix (ECM) proteins. The ECM mix contained from about 0.1 to about 3 mg/mL collagen, from about 0.1 to about 10 μg/mL fibronectin, and from about 0.1 to about 10 μg/mL of a basement membrane cocktail. The ECM mix was diluted with either a glycine buffer at pH 10 or a DMEM buffer at pH 7 depending on the cellular application.

### Example 10: Transfection Efficiency Using a Method of the Invention

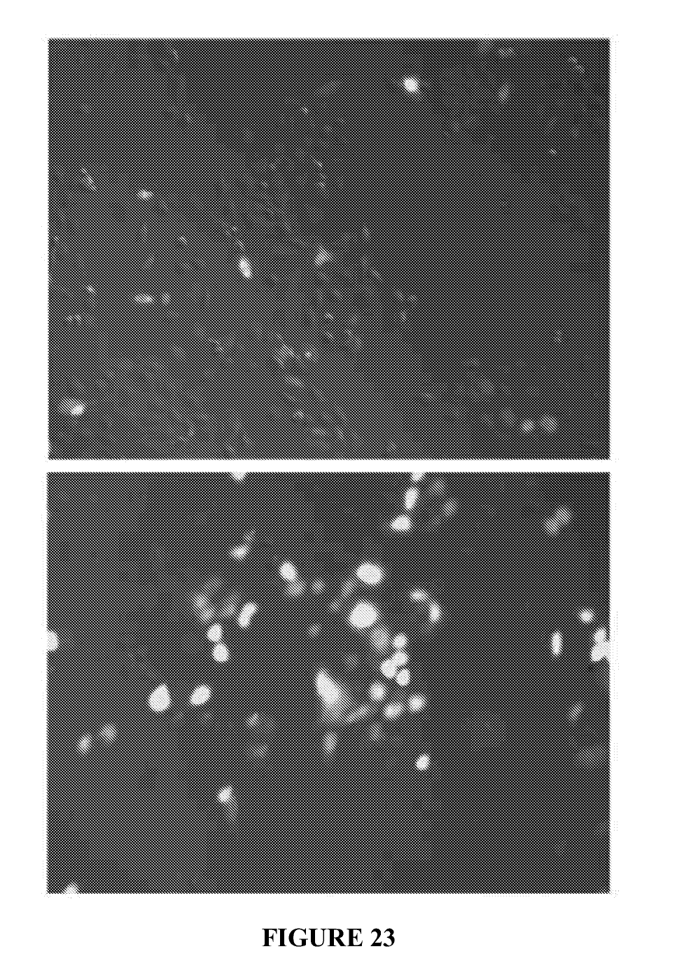

DU145 (human prostate cancer) cells were transfected with a GFP plasmid using electroporation. 5×10⁶ cells/mL were electroporated using a protocol of 1260 V for 20 ms twice with 50 ng DNA plasmid/μL of the cell resuspension. After transfection, the cells were split into separate 35 mm cell culture plates. One plate was placed in a standard CO₂ incubator, and the other plate was place in an incubator of the invention. The second plate's incubator was set to 1% O₂ and 2 PSIG. After 48 hours, the cells were fixed and imaged for GFP expression using a fluorescent microscope. The data shown in FIG. 23 indicate that cells incubated at low oxygen and high pressure showed higher transfection efficiency (lower panel) than those cells incubated at standard conditions (upper panel) as depicted by a greater number of bright cells.

### EMBODIMENTS

The following non-limiting embodiments provide illustrative examples of the invention, but do not limit the scope of the invention.

### Embodiment 1

A cell culture incubator, wherein the cell culture incubator comprises: a) an enclosed environmental chamber; and b) a control unit, wherein the control unit is operably linked to the enclosed environmental chamber, wherein the control unit comprises a computer program product comprising a computer-readable medium having computer-executable code encoded therein, the computer-executable code adapted to encode: (i) an oxygen level module, wherein the oxygen level module is encoded to regulate an oxygen level of the enclosed environmental chamber, wherein the oxygen level module is encoded to control the removal of oxygen in the enclosed environmental chamber to generate a hypoxic oxygen level within the enclosed environmental chamber; (ii) a pressure module, wherein the pressure module is encoded to regulate a pressure of the enclosed environmental chamber, wherein the pressure module controls the addition of gas to generate a positive pressure condition in the enclosed environmental chamber; (iii) a temperature module, wherein the temperature module is encoded to regulate a temperature of the enclosed environmental chamber; and (iv) a humidity module, wherein the humidity module is encoded to regulate a humidity of the enclosed environmental chamber, wherein each of the oxygen level, pressure, temperature, and humidity mimics an in vivo microenvironment for a cell, wherein the cell culture incubator reaches each of an instructed oxygen level, pressure, temperature, and humidity within about 20 minutes of receiving an input of each of the instructed oxygen level, pressure, temperature, and humidity.

### Embodiment 2

The cell culture incubator of embodiment 1, wherein the in vivo microenvironment is a tumor microenvironment.

### Embodiment 3

The cell culture incubator of any one of embodiments 1-2, wherein the cell is a stem cell.

### Embodiment 4

The cell culture incubator of any one of embodiments 1-2, wherein the cell is a cancer cell.

### Embodiment 5

The cell culture incubator of any one of embodiments 1-2, wherein the cell is a circulating tumor cell.

### Embodiment 6

The cell culture incubator of any one of embodiments 1-2, wherein the cell is an immune cell.

### Embodiment 7

The cell culture incubator of any one of embodiments 1-6, wherein the cell is obtained from a biological sample.

### Embodiment 8

The cell culture incubator of embodiment 7, wherein the biological sample is blood.

### Embodiment 9

The cell culture incubator of embodiment 7, wherein the biological sample is a tumor.

### Embodiment 10

The cell culture incubator of embodiment 7, wherein the biological sample is saliva.

### Embodiment 11

The cell culture incubator of embodiment 7, wherein the biological sample is a tissue.

### Embodiment 12

The cell culture incubator of any one of embodiments 1-11, wherein the control unit is user-controlled.

### Embodiment 13

The cell culture incubator of any one of embodiments 1-11, wherein the control unit is automated.

### Embodiment 14

The cell culture incubator of any one of embodiments 1-13, wherein the oxygen module is encoded to maintain a hypoxic oxygen level.

### Embodiment 15

The cell culture incubator of any one of embodiments 1-14, wherein the pressure module is encoded to maintain a positive pressure condition.

### Embodiment 16

The cell culture incubator of any one of embodiments 1-15, wherein the oxygen level module is encoded to maintain the oxygen level in the enclosed environmental chamber at about 0.1% to about 21%.

### Embodiment 17

The cell culture incubator of any one of embodiments 1-16, wherein the oxygen level module is encoded to maintain the oxygen level in the enclosed environmental chamber at about 2%.

### Embodiment 18

The cell culture incubator of any one of embodiments 1-17, wherein the oxygen level module is encoded to maintain the oxygen level in the enclosed environmental chamber at about 0.1%.

### Embodiment 19

The cell culture incubator of any one of embodiments 1-18, wherein the pressure module is encoded to maintain the pressure in the enclosed environmental chamber at from about 1 PSIG to about 5 PSIG.

### Embodiment 20

The cell culture incubator of any one of embodiments 1-19, wherein the humidity module is encoded to maintain the humidity in the enclosed environmental chamber at about 85%.

### Embodiment 21

The cell culture incubator of any one of embodiments 1-20, wherein the control unit further comprises a computer program product comprising a computer-readable medium having computer-executable code encoded therein, the computer-executable code adapted to encode a carbon dioxide module, wherein the carbon dioxide module is encoded to regulate a carbon dioxide level of the enclosed environmental chamber.

### Embodiment 22

The cell culture incubator of any one of embodiments 1-21, wherein the cell culture incubator further comprises a gas inlet controlled by the oxygen level module.

### Embodiment 23

The cell culture incubator of any one of embodiments 1-22, wherein the cell culture incubator further comprises a gas inlet controlled by the pressure module.

### Embodiment 24

The cell culture incubator of any one of embodiments 1-23, wherein the cell culture incubator further comprises a water humidity tray controlled by the humidity module.

### Embodiment 25

The cell culture incubator of any one of embodiments 1-24, wherein the cell culture incubator further comprises a heating element controlled by the temperature module.

### Embodiment 26

The cell culture incubator of any one of embodiments 1-25, wherein the cell culture incubator is configured to accept a cell culture plate.

1-26. (canceled)

27. A cell culture incubator, wherein the cell culture incubator comprises:
a) an enclosed environmental chamber; and
b) a control unit operably linked to the enclosed environmental chamber, wherein the control unit comprises a computer program product, wherein the computer program product comprises a computer-readable medium, wherein the computer-readable medium comprises a computer-executable code encoded, wherein the computer-executable code is adapted to instruct:
(i) an oxygen level module which receives an input to adjust an oxygen level in the enclosed environmental chamber to generate an instructed oxygen level within the environmental chamber wherein the instructed oxygen level is an instructed hypoxic oxygen level;
(ii) a pressure module which receives an input to adjust pressure in the enclosed environmental chamber to generate an instructed pressure level within the environmental chamber wherein the instructed pressure level is an instructed positive pressure level;
(iii) a temperature module which receives an input to adjust temperature in the enclosed environmental chamber to generate an instructed temperature within the environmental chamber; and
(iv) a humidity module which receives an input to adjust humidity in the enclosed environmental chamber to generate an instructed humidity level within the environmental chamber.

28. The cell culture incubator of claim 27, wherein:
a) the enclosed environmental chamber reaches the instructed hypoxic oxygen level within at least one hour of the oxygen level module receiving the input to adjust the oxygen level within the enclosed environmental chamber;
b) the enclosed environmental chamber reaches the instructed positive pressure level within at least one hour of the pressure module receiving the input to adjust the pressure level within the enclosed environmental chamber;
c) the enclosed environmental chamber reaches the instructed temperature within at least one hour of the temperature module receiving the input to adjust the temperature within the enclosed environmental chamber; and
d) the enclosed environmental chamber reaches the humidity level within at least one hour of receiving the input to adjust the oxygen level in the enclosed environmental chamber.

29. The cell culture incubator of claim 27, wherein:
a) the enclosed environmental chamber reaches the instructed hypoxic oxygen level within at least 20 minutes of the oxygen level module receiving the input to adjust the oxygen level within the enclosed environmental chamber;
b) the enclosed environmental chamber reaches the instructed positive pressure level within at least 20 minutes of the pressure module receiving the input to adjust the pressure level within the enclosed environmental chamber;
c) the enclosed environmental chamber reaches the instructed temperature within at least 20 minutes of the temperature module receiving the input to adjust the temperature within the enclosed environmental chamber; and
d) the enclosed environmental chamber reaches the humidity level within at least 20 minutes of receiving the input to adjust the oxygen level in the enclosed environmental chamber.

30. The cell culture incubator of claim 27, wherein the control unit is user-controlled.

31. The cell culture incubator of claim 27, wherein the control unit is automated.

32. The cell culture incubator of claim 27, wherein the input to adjust the oxygen level in the enclosed environmental chamber is within a range of about 0.1% to about 21% oxygen.

33. The cell culture incubator of 27, wherein the input to adjust the oxygen level in the enclosed environmental chamber is about 2% oxygen.

34. The cell culture incubator of claim 27, wherein the input to adjust the oxygen level in the enclosed environmental chamber is about 0.1% oxygen.

35. The cell culture incubator of claim 27, wherein the cell culture incubator further comprises a gas inlet that is controlled by the oxygen level module.

36. The cell culture incubator of claim 27, wherein the input to adjust the pressure level in the enclosed environmental chamber is at about 1 PSIG to about 5 PSIG.

37. The cell culture incubator of claim 27, wherein the cell culture incubator further comprises a gas inlet that is controlled by the pressure module.

38. The cell culture incubator of claim 27, wherein the input to adjust the humidity level in the enclosed environmental chamber is at about 85%.

39. The cell culture incubator of claim 27, wherein the cell culture incubator further comprises a water humidity tray that is controlled by the humidity module.

40. The cell culture incubator of claim 27, wherein the cell culture incubator further comprises a heating element that is controlled by the temperature module.

41. The cell culture incubator of claim 27, wherein the computer-executable code is adapted to further instruct a carbon dioxide module which receives an input to adjust a carbon dioxide level in the enclosed environmental chamber to generate an instructed carbon dioxide level within the environmental chamber.

42. The cell culture incubator of claim 27, wherein the computer-executable code is instructed to vary any one or more of the instructed oxygen level, the instructed pressure, the instructed temperature, and the instructed humidity within the enclosed environmental chamber as a function of time.

43. The cell culture incubator of claim 27, wherein the instructed oxygen level, the instructed pressure, the instructed temperature, and the instructed humidity are used to enrich a target cell population from a heterogeneous population of cells, wherein the heterogeneous population of cells is obtained from a biological sample.

44. The cell culture incubator of claim 43, wherein the biological sample is taken from a tumor, a blood sample, a saliva sample, or a tissue.

45. The cell culture incubator of claim 43, wherein the target cell population is circulating tumor cells (CTCs), cancer stem cells (CSCs), hematopoietic stem cells (HSCs), endothelial progenitor cells (EPCs), pre-cancerous cells, stem cells, fetal stem cells, undifferentiated stem cells, fetal cells, bone marrow cells, progenitor cells, foam cells, mesenchymal cells, epithelial cells, epithelial progenitor cells, endothelial cells, endometrial cells, trophoblasts, cancer cells, red blood cells, white blood cells, immune system cells, connective tissue cells, hepatocytes, neurons, or induced pluripotent stem (IPS) cells.

46. The cell culture incubator of claim 27, wherein the instructed oxygen level, the instructed pressure, the instructed temperature, and the instructed humidity simulate an in vivo microenvironment for a target subpopulation of cells.

47. The cell culture incubator of claim 46, wherein the in vivo microenvironment is a tumor microenvironment, a circulating tumor cell microenvironment, a cancer cell microenvironment, or an immune cell microenvironment.
